SEQUENCE LISTING (27 sequences)

SEQ ID NO: 1 - DNA, 64 bp, Artificial Sequence
  Synthetic Construct
tgctgatctgaaggccaatgacatgcgttttggccactgactgacgcatgtcaggccttcagat

SEQ ID NO: 2 - DNA, 64 bp, Artificial Sequence
  Synthetic Construct
cctgatctgaaggcctgacatgcgtcagtcagtggccaaaacgcatgtcattggccttcagatc

SEQ ID NO: 3 - DNA, 64 bp, Artificial Sequence
  Synthetic Construct
tgctgaactgatgatctgccgaggtcgttttggccactgactgacgacctcgggatcatcagtt

SEQ ID NO: 4 - DNA, 64 bp, Artificial Sequence
  Synthetic Construct
cctgaactgatgatcccgaggtcgtcagtcagtggccaaaacgacctcggcagatcatcagttc

SEQ ID NO: 5 - DNA, 64 bp, Artificial Sequence
  Synthetic Construct
tgctgtgcacttcgcggctcatgccagttttggccactgactgactggcatgacgcgaagtgca

SEQ ID NO: 6 - DNA, 64 bp, Artificial Sequence
  Synthetic Construct
cctgtgcacttcgcgtcatgccagtcagtcagtggccaaaactggcatgagccgcgaagtgcac

SEQ ID NO: 7 - DNA, 64 bp, Artificial Sequence
  Synthetic Construct
tgctgtgtgaattccctcttatgctcgttttggccactgactgacgagcataagggaattcaca

SEQ ID NO: 8 - DNA, 64 bp, Artificial Sequence
  Synthetic Construct
cctgtgtgaattcccttatgctcgtcagtcagtggccaaaacgagcataagagggaattcacac

SEQ ID NO: 9 - DNA, 22 bp, Artificial Sequence
  Synthetic Construct
gttccagactgttgacctcttt

SEQ ID NO: 10 - DNA, 20 bp, Artificial Sequence
  Synthetic Construct
ctgcgctttcttcataaacc

SEQ ID NO: 11 - DNA, 21 bp, Artificial Sequence
  Synthetic Construct
atgggaagggaaggacatggc

SEQ ID NO: 12 - DNA, 21 bp, Artificial Sequence
  Synthetic Construct
gctgggctttcctgtgaaacc

SEQ ID NO: 13 - DNA, 25 bp, Artificial Sequence
  Synthetic Construct
atacactctcttctctcacgctgtg

SEQ ID NO: 14 - DNA, 22 bp, Artificial Sequence
  Synthetic Construct
aagagcaccttccatgacagac

SEQ ID NO: 15 - DNA, 22 bp, Artificial Sequence
  Synthetic Construct
cctgatgaattcaaaccgaatc

SEQ ID NO: 16 - DNA, 25 bp, Artificial Sequence
  Synthetic Construct
aggagaggtccatttgtagaagtga

SEQ ID NO: 17 - DNA, 23 bp, Artificial Sequence
  Synthetic Construct
aaatgcttggttcaccagtggat

SEQ ID NO: 18 - DNA, 24 bp, Artificial Sequence
  Synthetic Construct
cactgccattttagctccttcttg

SEQ ID NO: 19 - DNA, 23 bp, Artificial Sequence
  Synthetic Construct
ttcagacaggatgttgacaatgc

SEQ ID NO: 20 - DNA, 23 bp, Artificial Sequence
  Synthetic Construct
ggatttcctcttcgtggagtttc

SEQ ID NO: 21 - DNA, 25 bp, Artificial Sequence
  Synthetic Construct
cgagagtaaacctgaagctgaagag

SEQ ID NO: 22 - DNA, 22 bp, Artificial Sequence
  Synthetic Construct
gatgcaggtacagtcccagatc

SEQ ID NO: 23 - DNA, 23 bp, Artificial Sequence
  Synthetic Construct
acagcctcaagatcatcagcaat

SEQ ID NO: 24 - DNA, 23 bp, Artificial Sequence
  Synthetic Construct
atggactgtggtcatgagtcctt

SEQ ID NO: 25 - DNA, 21 bp, Artificial Sequence
  Synthetic Construct
gagcataagagggaattcaca

SEQ ID NO: 26 - DNA, 21 bp, Artificial Sequence
  Synthetic Construct
gagcataaaagagaattcaca

SEQ ID NO: 27 - protein, 7 aa, Artificial Sequence
  Synthetic Construct
EHKREFT

(19) United States
(12) Patent Application Publication
(10) Pub. No.: US 2010/0069470 A1
(43) Pub. Date: Mar. 18, 2010

(54) IDENTIFICATION OF PROTEIN BIOMARKERS ASSOCIATED WITH LYMPH NODE METASTASIS IN COLORECTAL CANCER

(76) Inventors: Ying LIN, Haizhu District Guangzhou (CN); William S. DYNAN, Martinez, GA (US); Jeffrey R. LEE, Martinez, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 12/551,168

(22) Filed: Aug. 31, 2009

Related U.S. Application Data
(60) Provisional application No. 61/190,504, filed on Aug. 29, 2008.

(51) Int. Cl. A61K 31/7052; G01N 33/68; C12Q 1/02; G01N 33/53; A61P 35/00

(52) U.S. Cl. 514/44 R; 436/86; 435/29; 436/501

(57) ABSTRACT

Disclosed herein are compositions and methods relating to the diagnosis, prognosis, and treatment of colorectal cancer. The compositions and methods generally relate to the differential expression of transgelin in colorectal cancer cells with metastatic potential.

### CROSS-REFERENCE TO RELATED APPLICATION

This application claims the benefit of U.S. Provisional Patent Application Ser. No. 61/190,504, filed Aug. 29, 2008, the entirety of which is hereby incorporated herein by reference for all purposes.

### STATEMENT REGARDING FEDERALLY SPONSORED RESEARCH

This invention was made with government support under Grant R33 CA95941 awarded by the National Institutes of Health. The government has certain rights in the invention.

### BACKGROUND OF THE INVENTION

Colorectal cancer (CRC) is the third leading cause of cancer death in both men and women in the United States [Jemal A, et al. 2008]. Metastatic dissemination of primary tumors is responsible for 90% of all CRC deaths [Christofori G. 2006].

The tumor-node-metastasis (TNM) staging system is a primary tool for patient management. Detection of positive lymph nodes separates stage I/II from stage III CRC, and is often a key factor in determining patient management. The presence of cancer cells in lymph nodes is the watershed event that separates TNM stage I/II disease, where the standard of care is complete surgical resection without adjuvant treatment, and stage III disease, which is unlikely to be curable by resection alone [Compton C C. 2007]. Lymph node status can, however, only be ascertained post-surgically. Moreover, it is strongly influenced by the number of nodes examined [Compton C C. 2007]. In one study, nodal metastasis was found in 22% of cases where less than 15 nodes were harvested, but in 85% where 15 or more were examined [Goldstein N S, et al. 2002]. Recovery and examination of a greater number of lymph nodes confers a clear survival advantage [Goldstein N S, et al. 1996]. Mathematical modeling suggest that there is a better-than-even chance of missing a hypothetical positive node even if 18 nodes are examined [Goldstein N S, et al. 1996]. Thus, patients may easily be understaged by this method.

The advantage of accurate staging is likely to grow further as better therapies become available for the treatment of metastatic CRC [Saletti P. and Cavalli F. 2006]. A little more than a decade ago, only one agent, 5-fluorouracil, was available for adjuvant therapy. The subsequent advent of combination therapies resulted in a doubling of the response rate and median overall survival [Saletti P. and Cavalli F. 2006]. The recent introduction of the anti-angiogenesis agent, bevacizumab, for stage III/IV CRC has further increased the effectiveness of adjuvant treatment [Grothey A. and Ellis L M. 2008]. The availability of effective therapies for metastatic CRC underscores the potential benefit to be gained from better, molecularly-based indicators of metastatic potential. Tests based on such indicators could identify stage II patients who would most likely benefit from intensive surveillance or adjuvant therapy following surgical resection.

Accordingly, it can be seen that needs exist for improved methods for detecting, staging, and treating colorectal cancer. It is to the provision of meeting these and other needs that the present invention is primarily directed.

### SUMMARY OF THE INVENTION

In example embodiments, the present invention provides compositions and methods for determining the metastatic potential of a colorectal cancer in a subject, the method comprising assaying for the levels of transgelin in a sample from the subject, wherein an increase in transgelin levels in the sample compared to a control level is an indication that the subject is at high risk of developing metastatatic colorectal cancer.

Also provided is a method of selecting a therapy for a subject diagnosed with a colorectal cancer, the method comprising assaying for the levels of transgelin in a sample from the subject, wherein an increase in transgelin levels in the sample compared to a control level is an indication that the therapy selected comprises surgical resection and adjuvant treatment.

Also provided is a method of treating colorectal cancer in a subject, comprising administering to the subject an inhibitor of transgelin activity.

Also provided is a method of identifying an agent for use in treating colorectal cancer, comprising contacting a sample comprising transgelin with a candidate agent and assaying for transgelin activity in the sample, wherein a decrease in transgelin activity in the sample is an indication that the candidate agent is an effective agent for use in inhibiting the metastasis of colorectal cancer.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1 shows proteomic profiling of CRCs stratified by node status. FIG. 1A shows the relationship between biomarkers identified in different comparisons. FIG. 1B shows differential expression of transgelin. Top: representative two-dimensional gel images; bottom: three-dimensional representation. Arrowheads indicate transgelin. FIG. 1C shows the standardized log-transformed abundance of transgelin in node-negative and node-positive CRC. FIG. 1D shows receiver operating characteristic (ROC) curve for transgelin in prediction of node status.

FIG. 2 are Illustrations of hematoxylin and eosin staining and immunohistochemistry of antitransgelin on CRC TMAs.

FIG. 3 shows MicroRNA-mediated knockdown and rescue of TAGLN in CRC cells. FIG. 3A shows immunoblot analysis of HCT116 cells after transient transfection of control miRNA plasmid and four different TAGLN miRNA plasmids. FIG. 3B shows Immunoblot analysis of HCT116 and SW480 cells with stable expression of control miRNA or TAGLN miRNA-4. FIG. 3C shows real-time PCR analysis of TAGLN and TAGLN3 mRNA expression in HCT116 and SW480 cells with stable expression of control miRNA (HCT116^{CTRL}, SW480^{CTRL}) and TAGLN miRNA-4 (HCT116^{TAGLN-KD}, SW480^{TAGLN-}KD). Gene expression was normalized to GAPDH. Data are expressed as mean±SD from three independent experiments; **P<0.01. FIG. 3D shows fluorescence microscopy of HCT116 and SW480 stably transfected cells (original magnification, ×63; white bar, 20 μm). Panels show transgelin immunostaining, EmGFP transfection marker, DAPI DNA staining, and a merged image as indicated. FIG. 3E shows TAGLN miRNA target (SEQ ID NO:25) and rescue sequences (SEQ ID NO:26); also shown is the translation product (SEQ ID NO:27). FIG. 3F shows immunoblot analysis of HCT116^{TAGLN-KD} cells after transient transfection with empty vector or TAGLN rescue plasmid.

FIG. 4 shows effects of TAGLN silencing on HCT116 and SW480 cell invasion, survival, and anoikis. FIG. 4A shows invasion assay. Representative images of Transwell filters indicating HCT116_{CTRL} and HCT116^{TAGLN-KD} invasion are shown. Bar graphs show comparison of invasion capacities of HCT116 and SW480 stably transfected cells (left) and HCT116^{TAGLN-KD} and SW480^{TAGLN-KD} cells after transient transfection with 1 μg of empty vector or TAGLN rescue plasmid (right). FIG. 4B shows clonogenic survival assay. Representative images of HCT116^{CTRL} and HCT116^{TAGLN-KD} cells. Graph shows the number of colonies formed 10 and 14 days after plating HCT116 and SW480 stably transfected cells, respectively. FIG. 4C shows anoikis-induced apoptosis measured by Annexin V and propidium iodide staining and flow cytometry analysis at 72 hours after plating HCT116 and SW480 stably transfected cells on polyHEMA-coated culture dishes. Anoikis was also assessed by counting the viable cells at 24 hours after replating the anoikis-induced cells on regular culture dishes. Values for control cells were considered 100%, any differences are expressed relative to this value. Data are expressed as mean±SD from three independent experiments; **P<0.01.

FIG. 5 shows EMT marker genes regulated by TAGLN. The mRNA levels of the epithelial marker genes occludin (OCCL) and mesenchymal marker genes vimentin (VIM) and fibronectin 1 (FN1) in HCT116^{CTRL} and HCT116^{TAGLN-KD} cells (FIG. 5A) and in SW480^{CTRL} and SW480^{TAGLN-KD} cells (FIG. 5B). Gene expression was measured by real-time RT-PCR and normalized to GAPDH. Data are expressed as mean±SD from three independent experiments; **P<0.01, *P<0.05.

FIG. 6 shows histologic appearance of tumors in CB.17 mice. Same specimens at three magnifications (note scale bars, dimension indicated in μm).

FIG. 7 shows STRING tool output reveals directly TAGLN-connected genes and 10 additional indirectly connected genes. Key indicates type of supporting evidence.

FIG. 8 shows co-regulated proteins and their predicted partners.

### DETAILED DESCRIPTION

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Disclosed are materials, compositions, and components that can be used for, can be used in conjunction with, can be used in preparation for, or are products of the disclosed composition(s) and method(s). These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C is disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F is specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed, it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the composition(s) and method(s) described herein. Such equivalents are intended to be encompassed by the appended claims.

It is understood that the disclosed composition(s) and method(s) are not limited to the particular methodology, protocols, and reagents described as these may vary. It is also to be understood that the terminology used herein is for the purpose of describing particular embodiments only and is not intended to limit the scope of the present invention which will be limited only by the appended claims.

### A. DEFINITIONS

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

“Optional” or “optionally” means that the subsequently described event, circumstance, or material may or may not occur or be present, and that the description includes instances where the event, circumstance, or material occurs or is present and instances where it does not occur or is not present.

Throughout the description and claims of this specification, the word “comprise” and variations of the word, such as “comprising” and “comprises,” means “including but not limited to” and is not intended to exclude, for example, other additives, components, integers or steps.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As used herein, the term “subject” means any individual who is the target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), the specified ligand or antibody binds to its particular “target” (e.g. an antibody specifically binds to its epitope) and does not bind in a significant amount to other proteins present in the sample or to other proteins to which the ligand or antibody may come in contact in an organism. Generally, a first molecule that “specifically binds” a second molecule has a binding affinity greater than about 10⁵ (e.g., 10⁶, 10⁷, 10⁸, 10⁹, 10¹⁰, 10¹¹, and 10¹² or more) moles/liter for that second molecule.

### B. METHODS

1. Diagnostic/Prognostic Methods

The majority (95%) of human CRC cases arise spontaneously via the acquisition of spontaneous mutations in oncogenes and tumor suppressor genes. Although the clinical, morphological, and molecular features of CRC are heterogeneous, its pathogenesis is most commonly characterized by histopathological changes known as the adenoma-carcinoma sequence [Vogelstein B., et al. 1988]. The sequence of events typically begins with disruption of the adenomatous polyposis coli (APC)/β-catenin pathway. Disruption of the APC/β-catenin pathway creates self-sufficiency in growth signals, which is one of the “hallmarks of cancer” [Hanahan D. and Weinberg RA. 2000]. Other “hallmarks of cancer” include insensitivity to anti-growth signals, self-sufficiency in growth signaling, and limitless growth potential. There are six additional regulatory pathways that are commonly disrupted in CRC. These include the retinoblastoma (Rb) pathway, which regulates entry into S phase of the cell cycle; the TGF-β/SMAD pathway, which mediates anti-growth signaling in epithelial cells, and the phospatidyl inositol-3 kinase (PI-3K) pathway, which mediates growth promoting gene (GPG) transcription and translation. They also include receptor tyrosine kinase (RTK) pathways, which control expression of GPGs, the p53 pathway, which mediates cell cycle arrest and apoptosis, and the apoptosis pathway, which can mediate selective elimination of transformed cells.

Metastatic dissemination, rather than the primary tumor, is responsible for 90% of CRC deaths [Christofori G. 2006]. As with progression from adenoma to carcinoma, the acquisition of metastatic potential is an evolutionary process in which cancer cells and their microenvironment undergo progressive alterations [Chiang A C and Massague J. 2008]. The genetics of metastasis are not as thoroughly understood as those of tumor initiation and growth [Chiang A C and Massague J. 2008]. It is believed, however, that metastasis requires engagement of a program of genetic changes known as the epithelial-mesenchymal transition (EMT). The EMT is controlled in part by a set of transcription factors (Snail, Slug, ZEB-1, ZEB-2/SIP-1, and Twist) that silence the gene for E-cadherin, an epithelial adhesion protein. Elevated Snail expression has been reported in 78% of CRC cases, and elevated Slug expression in 37% [Natalwala A., et al. 2008].

Loss of heterozygosity (LOH) at chromosome 8p has also frequently been detected in advanced CRC [Diep C B., et al. 2006; Zhou W., et al. 2002]. In other cancers (prostate and head and neck cancer) 8p LOH is significantly associated with node-positive status, although the correlation is not as high in CRC [Oba K, et al. 2001). Recent work suggests that one of the important genes in the affected region may be CSMD1, a transmembrane protein thought to be involved in cell migration [Farrell C. 2008].

The growing understanding of the genetics of advanced cancer raises the prospect of developing a clinical test, based on molecular markers that could be used to guide patient management. Such a test could augment TNM staging, in particular to avoid under-staging patients with undetected positive nodes. In breast cancer, tests based on expression profiling of the primary tumor have demonstrated their prognostic value [Jezequel P., et al. 2008]. However, there is presently no comparably useful test that can be performed on a resected primary CRC.

Proteins are the ultimate effectors of gene function, and proteomic profiling of tumor samples has the potential to identify practical biomarkers that could form the basis of a test for metastatic potential. Samples for clinical proteomics come from three sources: serum, other accessible body fluids, and tissue. Proteomic analysis of serum and other accessible body fluids have been the focus of much attention because of the attractiveness of noninvasive screening test. Tissue samples, by contrast, are the material of choice for establishing prognosis in patients who have already been diagnosed. Extraction provides access to a variety of intracellular regulatory proteins, such as regulatory kinases or transcription factors, which would not routinely be present in serum or other body fluids. Tumor samples, obtained through biopsy or surgical resection, contain a wealth of information.

Tumor samples contain a mixture of normal tissue, viable cancer cells, stroma, and necrotic regions. Laser capture microdissection (LCM) provides a means to obtain pure cell populations and is preferable to analysis of bulk samples. Stromal contamination is particularly an issue when screening for biomarkers associated with metastatic potential. This is because stromal fibroblasts already contain mesenchymal markers, and their presence potentially confounds detection of changes in the level of these markers in the cancer cells themselves.

Twelve reports have described proteomic profiling of CRC or premalignant adenomas [reviewed in Lin Y., et al. 2009]. Seven studies, all using two dimensional electrophoresis (2-DE) for initial protein separation, identified numerous differentially expressed proteins including transcription regulators, signal transduction and cytoskeletal proteins, molecular chaperones, protein synthesis factors, metabolic enzymes, apoptosis-associated proteins, and a proteoglycan (mimecan). One study used a unique, gene- and antibody-based approach that avoids biases inherent in 2-DE [Madoz-Gurpide J., et al. 2006]. Two studies used novel methodology based on direct mass spectrometry (MS) analysis of tissue sections, which provides spatially-resolved images of in situ protein abundance in tumor versus normal areas.

Only two of the twelve studies specifically sought to identify biomarkers to discriminate tumors derived from node-positive versus node-negative patients. One study used 2-DE to analyze bulk (non-microdissected) CRC tissue [Pei H. et al. 2007]. This study identified four candidate biomarkers: increased HSP B1 (HSP27), glutathione-S-transferase, and annexin II, and decreased liver-fatty acid binding protein. The association appeared to be clinically significant in follow-up immunoblotting and tissue microarray studies. However, the three proteins described as up-regulated in metastatic tissues have no obvious mechanistic connection to tissue invasion or other hallmarks of metastasis, and they have also been found to be up-regulated in many other studies of cancer versus normal tissue (i.e., without regard to metastatic potential), so their ultimate utility is uncertain.

The herein disclosed study comparing node-positive and node-negative CRC patients incorporated technical and other refinements, including (a) a larger patient cohort, (b) LCM sampling, (c) precise quantification based on multiplex labeling (median coefficient of technical variation=10%), and (d) a different statistical approach that allowed accurate ranking of candidate proteomic features [Lin Y. et al. 2009]. The results differed from the earlier study: the top-ranked biomarker was transformation-sensitive actin gelling protein, or transgelin (gene symbol, TAGLN). As disclosed herein, biological functions of transgelin do appear to be connected with hallmarks of metastasis, including motility, invasiveness, resistance to anoikis, and engagement of the epithelial-to-mesenchymal program of gene expression. Furthermore, artificial manipulation of TAGLN expression dramatically alters the pattern of tumor spread in a xenograft model.

Thus, provide herein is a method of determining the metastatic potential of a cancer in a subject. In some aspects, the cancer is one wherein epithelial-mesenchymal transition (EMT) is necessary for metastasis. Thus, in some aspects, the cancer is a colorectal cancer in a subject.

The method can comprise assaying for the levels of transgelin in a sample from the subject. In some aspects, an increase in transgelin levels in the sample compared to a control level is an indication that the cancer in the subject is metastatic.

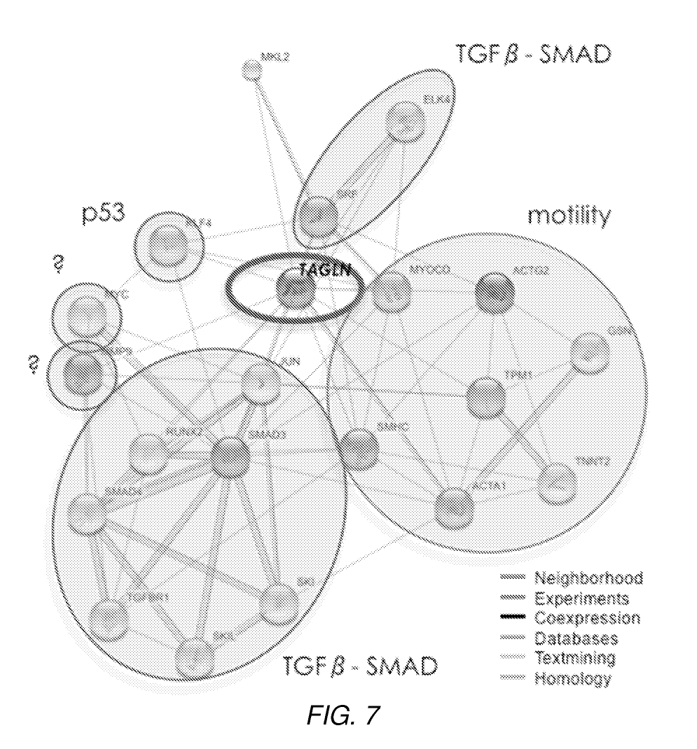

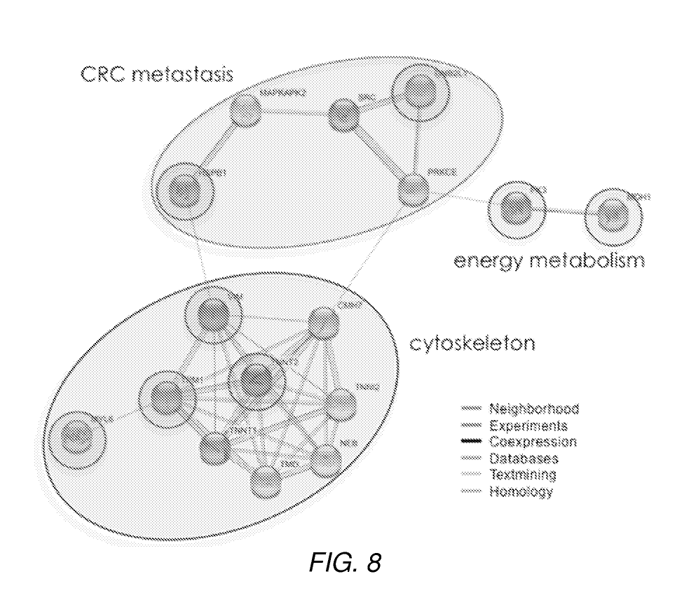

Other biomarkers have been identified herein that can be used alone or in combination with transgelin in the herein disclosed methods. For example, additional biomarkers that are differentially expressed in metastatic colorectal carcinoma cells are shown in Table 2 and can be used in the disclosed methods. Likewise, the results shown in FIG. 7 and FIG. 8 identify additional biomarkers for use in the disclosed methods.

Thus, the method can comprise assaying for the levels of a biomarker in a sample from the subject. In some aspects, an increase in transgelin levels in the sample compared to a control level is an indication that the cancer in the subject is metastatic. Thus, in some aspects, the disclosed method can further comprise assaying for the levels of any one or more of the biomarkers disclosed in Table 2. Thus, in some aspects, the disclosed method can further comprise assaying for the levels of ACTA1, ACTG2, CMH7, ELK4, GSN, HSPB1, JUN, KLF4, MAPKAPK2, MLK2, MMP9, MOH1, MYC, MYL6, MYOCD, NEB, PK3, PRKCE, RACK1, RUNX2, SKI, SKIL, SMAD3, SMAD4, SMHC, SRC, SRF, TGFBR1, TMD, TNNT2, TNNT2, TPM1, or VIM. In some aspects, the disclosed method can further comprise assaying for the levels of vimentin, myosin light chain 6, RACK1, serum amyloid P, cytosolic malate dehydrogenase, tropomyosin 1, Hsp 27, pyruvate kinase, aldehyde dehydrogenase, or isocitrate dehydrogenase.

Moreover, in some aspects, the disclosed biomarkers can be used in the disclosed methods instead of transgelin. Thus, in some aspects, reference in the herein disclosed methods to “transgenin” is hereby also a reference to the proteins in Table 2 shown to be differentially expressed in metastatic colorectal carcinoma cells. In some aspects, reference in the herein disclosed methods to “transgenin” is hereby also a reference to ACTA1, ACTG2, CMH7, ELK4, GSN, HSPB1, JUN, KLF4, MAPKAPK2, MLK2, MMP9, MOH1, MYC, MYL6, MYOCD, NEB, PK3, PRKCE, RACK1, RUNX2, SKI, SKIL, SMAD3, SMAD4, SMHC, SRC, SRF, TGFBR1, TMD, TNNT2, TNNT2, TPM1, or VIM.

Further, combinations of each and every biomarker disclosed herein is contemplated for use in the disclosed methods.

As disclosed herein, TNM staging when used alone can result in under-staging patients with undetected positive nodes. In some aspects, whether cancer cells are detected depends on the number of nodes examined. Thus, in some aspects of the method, cancer cells were not previously detected in the lymph nodes of the subject. In some aspects, the sample is a tumor biopsy.

Also disclosed is a method of selecting a therapy for a subject diagnosed with a cancer, the method comprising assaying for the levels of transgelin in a sample from the subject, wherein an increase in transgelin levels in the sample compared to a control level is an indication that the therapy selected comprises surgical resection and adjuvant treatment. In some aspects, the cancer is one wherein epithelial-mesenchymal transition (EMT) is necessary for metastasis. Thus, in some aspects, the cancer is a colorectal cancer in a subject.

i. Immunoassay

In some aspects, the method is an immunoassay. Immunoassays, in their most simple and direct sense, are binding assays involving binding between antibodies and antigen. Thus, in some aspects, the method comprises detecting transgelin using an antibody that specifically binds transgelin, such as human transgelin. Antibodies that specifically bind human transgelin (TAGLN) are commercially available and can be produced using routine skill.

Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

A modifier unit such as a radionuclide can be incorporated into or attached directly to any of the compounds described herein by halogenation. In another aspect, the radionuclide can be attached to a linking group or bound by a chelating group, which is then attached to the compound directly or by means of a linker. Radiolabeling techniques such as these are routinely used in the radiopharmaceutical industry.

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

ii. Nucleic Acid Detection

In some aspects, the method comprises detecting transgelin using a primer or probe that selectively binds TAGLN mRNA.

A number of widely used procedures exist for detecting and determining the abundance of a particular mRNA in a total or poly(A) RNA sample. For example, specific mRNAs can be detected using Northern blot analysis, nuclease protection assays (NPA), in situ hybridization, or reverse transcription-polymerase chain reaction (RT-PCR).

In theory, each of these techniques can be used to detect specific RNAs and to precisely determine their expression level. In general, Northern analysis is the only method that provides information about transcript size, whereas NPAs are the easiest way to simultaneously examine multiple messages. In situ hybridization is used to localize expression of a particular gene within a tissue or cell type, and RT-PCR is the most sensitive method for detecting and quantitating gene expression.

Northern analysis presents several advantages over the other techniques. The most compelling of these is that it is the easiest method for determining transcript size, and for identifying alternatively spliced transcripts and multigene family members. It can also be used to directly compare the relative abundance of a given message between all the samples on a blot. The Northern blotting procedure is straightforward and provides opportunities to evaluate progress at various points (e.g., intactness of the RNA sample and how efficiently it has transferred to the membrane). RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

The Nuclease Protection Assay (NPA) (including both ribonuclease protection assays and S1 nuclease assays) is an extremely sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. Solution hybridization is typically more efficient than membrane-based hybridization, and it can accommodate up to 100 μg of sample RNA, compared with the 20-30 μg maximum of blot hybridizations. NPAs are also less sensitive to RNA sample degradation than Northern analysis since cleavage is only detected in the region of overlap with the probe (probes are usually about 100-400 bases in length).

NPAs are the method of choice for the simultaneous detection of several RNA species. During solution hybridization and subsequent analysis, individual probe/target interactions are completely independent of one another. Thus, several RNA targets and appropriate controls can be assayed simultaneously (up to twelve have been used in the same reaction), provided that the individual probes are of different lengths. NPAs are also commonly used to precisely map mRNA termini and intron/exon junctions.

In situ hybridization (ISH) is a powerful and versatile tool for the localization of specific mRNAs in cells or tissues. Unlike Northern analysis and nuclease protection assays, ISH does not require the isolation or electrophoretic separation of RNA. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents.

RT-PCR has revolutionized the study of gene expression. It is now theoretically possible to detect the RNA transcript of any gene, regardless of the scarcity of the starting material or relative abundance of the specific mRNA. In RT-PCR, an RNA template is copied into a complementary DNA (cDNA) using a retroviral reverse transcriptase. The cDNA is then amplified exponentially by PCR. As with NPAs, RT-PCR is somewhat tolerant of degraded RNA. As long as the RNA is intact within the region spanned by the primers, the target will be amplified.

Relative quantitative RT-PCR involves amplifying an internal control simultaneously with the gene of interest. The internal control is used to normalize the samples. Once normalized, direct comparisons of relative abundance of a specific mRNA can be made across the samples. It is crucial to choose an internal control with a constant level of expression across all experimental samples (i.e., not affected by experimental treatment). Commonly used internal controls (e.g., GAPDH, β-actin, cyclophilin) often vary in expression and, therefore, may not be appropriate internal controls. Additionally, most common internal controls are expressed at much higher levels than the mRNA being studied. For relative RT-PCR results to be meaningful, all products of the PCR reaction must be analyzed in the linear range of amplification. This becomes difficult for transcripts of widely different levels of abundance.

Competitive RT-PCR is used for absolute quantitation. This technique involves designing, synthesizing, and accurately quantitating a competitor RNA that can be distinguished from the endogenous target by a small difference in size or sequence. Known amounts of the competitor RNA are added to experimental samples and RT-PCR is performed. Signals from the endogenous target are compared with signals from the competitor to determine the amount of target present in the sample.

2. Screening Methods

Also provided herein is a method of identifying an agent for use in treating cancer, comprising contacting a sample comprising transgelin with a candidate agent and assaying for transgelin activity in the sample, wherein a decrease in transgelin activity in the sample is an indication that the candidate agent is an effective agent for use in treating cancer.

As used herein, the ability of an agent to “treat” cancer includes the ability of the therapeutic to prevent or reduce the onset of metastasis by the cancer. Thus, the agent of the disclosed methods can in some aspects re treat cancer by reducing the severity of the cancer.

In some aspects, the cancer of the disclosed method is one wherein epithelial-mesenchymal transition (EMT) is necessary for metastasis. Thus, in some aspects, the cancer is a colorectal cancer in a subject.

As used herein, “activities” of a protein include, for example, transcription, translation, intracellular translocation, secretion, phosphorylation by kinases, cleavage by proteases, homophilic and heterophilic binding to other proteins, ubiquitination. Thus, the transgelin activity being assayed can be the levels of transgelin in the sample.

The methods can be cell-based or cell-free assays. Thus, in some aspects, the method comprises a cell-based assay. For example, in some aspects, the cell of the cell-based assay is a colorectal cancer cell line.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-cancer effects should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that inhibits transgelin. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

3. Treatment Methods

Also disclosed is a method of treating colorectal cancer in a subject, comprising administering to the subject an inhibitor of transgelin activity. The transgelin inhibitor can be any molecule, protein, nucleic acid, or other composition known or discovered inhibit the activity of transgelin in a colorectal cancer cell.

i. Functional Nucleic Acids

In some aspects, the transgelin inhibitor is a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of transgelin or the genomic DNA of transgelin or they can interact with the polypeptide transgelin. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d})less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with K_{d}'s from the target molecule of less than 10-12 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat) hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences for transgelin.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for transgelin

MicroRNAs (miRNA or pRNA) are single-stranded RNA molecules of 21-23 nucleotides in length, which regulate gene expression. miRNAs are encoded by genes from whose DNA they are transcribed but miRNAs are not translated into protein (i.e. they are non-coding RNAs); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression. Disclosed herein are any miRNA designed as described above based on the sequences for transgelin.

ii. Pharmaceutical Compositions

The transgelin inhibitor disclosed herein can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

iii. Therapeutic Administration

The herein disclosed transgelin inhibitors, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained. See, e.g., U.S. Pat. No. 3,610,795, which is incorporated by reference herein.

The compositions disclosed herein may be administered prophylactically to patients or subjects who are at risk for colorectal cancer. Thus, the method can further comprise identifying a subject at risk metastatic colorectal cancer prior to administration of the herein disclosed transgelin inhibitors.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

iv. Combination Therapies

Provided herein is a composition that comprises a transgelin inhibitor and any known or newly discovered substance that can be administered to the site of a colorectal cancer.

Numerous anti-cancer (antineoplastic) drugs are available for combination with the present method and compositions. Antineoplastic drugs include Acivicin, Aclarubicin, Acodazole Hydrochloride, AcrQnine, Adozelesin, Aldesleukin, Altretamine, Ambomycin, Ametantrone Acetate, Aminoglutethimide, Amsacrine, Anastrozole, Anthramycin, Asparaginase, Asperlin, Azacitidine, Azetepa, Azotomycin, Batimastat, Benzodepa, Bicalutamide, Bisantrene Hydrochloride, Bisnafide Dimesylate, Bizelesin, Bleomycin Sulfate, Brequinar Sodium, Bropirimine, Busulfan, Cactinomycin, Calusterone, Caracemide, Carbetimer, Carboplatin, Carmustine, Carubicin Hydrochloride, Carzelesin, Cedefingol, Chlorambucil, Cirolemycin, Cisplatin, Cladribine, Crisnatol Mesylate, Cyclophosphamide, Cytarabine, Dacarbazine, Dactinomycin, Daunorubicin Hydrochloride, Decitabine, Dexormaplatin, Dezaguanine, Dezaguanine Mesylate, Diaziquone, Docetaxel, Doxorubicin, Doxorubicin Hydrochloride, Droloxifene, Droloxifene Citrate, Dromostanolone Propionate, Duazomycin, Edatrexate, Eflomithine Hydrochloride, Elsamitrucin, Enloplatin, Enpromate, Epipropidine, Epirubicin Hydrochloride, Erbulozole, Esorubicin Hydrochloride, Estramustine, Estramustine Phosphate Sodium, Etanidazole, Ethiodized Oil I 131, Etoposide, Etoposide Phosphate, Etoprine, Fadrozole Hydrochloride, Fazarabine, Fenretinide, Floxuridine, Fludarabine Phosphate, Fluorouracil, Flurocitabine, Fosquidone, Fostriecin Sodium, Gemcitabine, Gemcitabine Hydrochloride, Gold Au 198, Hydroxyurea, Idarubicin Hydrochloride, Ifosfamide, Ilmofosine, Interferon Alfa-2a, Interferon Alfa-2b, Interferon Alfa-n1, Interferon Alfa-n3, Interferon Beta-Ia, Interferon Gamma-Ib, Iproplatin, Irinotecan Hydrochloride, Lanreotide Acetate, Letrozole, Leuprolide Acetate, Liarozole Hydrochloride, Lometrexol Sodium, Lomustine, Losoxantrone Hydrochloride, Masoprocol, Maytansine, Mechlorethamine Hydrochloride, Megestrol Acetate, Melengestrol Acetate, Melphalan, Menogaril, Mercaptopurine, Methotrexate, Methotrexate Sodium, Metoprine, Meturedepa, Mitindomide, Mitocarcin, Mitocromin, Mitogillin, Mitomalcin, Mitomycin, Mitosper, Mitotane, Mitoxantrone Hydrochloride, Mycophenolic Acid, Nocodazole, Nogalamycin, Ormaplatin, Oxisuran, Paclitaxel, Pegaspargase, Peliomycin, Pentamustine, Peplomycin Sulfate, Perfosfamide, Pipobroman, Piposulfan, Piroxantrone Hydrochloride, Plicamycin, Plomestane, Porfimer Sodium, Porfiromycin, Prednimustine, Procarbazine Hydrochloride, Puromycin, Puromycin Hydrochloride, Pyrazofurin, Riboprine, Rogletimide, Safmgol, Safingol Hydrochloride, Semustine, Simtrazene, Sparfosate Sodium, Sparsomycin, Spirogermanium Hydrochloride, Spiromustine, Spiroplatin, Streptonigrin, Streptozocin, Strontium Chloride Sr 89, Sulofenur, Talisomycin, Taxane, Taxoid, Tecogalan Sodium, Tegafur, Teloxantrone Hydrochloride, Temoporfin, Teniposide, Teroxirone, Testolactone, Thiamiprine, Thioguanine, Thiotepa, Tiazofurin, Tirapazamine, Topotecan Hydrochloride, Toremifene Citrate, Trestolone Acetate, Triciribine Phosphate, Trimetrexate, Trimetrexate Glucuronate, Triptorelin, Tubulozole Hydrochloride, Uracil Mustard, Uredepa, Vapreotide, Verteporfin, Vinblastine Sulfate, Vincristine Sulfate, Vindesine, Vindesine Sulfate, Vinepidine Sulfate, Vinglycinate Sulfate, Vinleurosine Sulfate, Vinorelbine Tartrate, Vinrosidine Sulfate, Vinzolidine Sulfate, Vorozole, Zeniplatin, Zinostatin, Zorubicin Hydrochloride.

Other anti-neoplastic compounds include: 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, atrsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorines, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexifosfamide, dexrazoxane, dexverapamil, diaziquone, didemnin B, didox, diethylnorspermine, dihydro-5-azacytidine, dihydrotaxol, 9-dioxamycin, diphenyl spiromustine, docosanol, dolasetron, doxifluridine, droloxifene, dronabinol, duocannycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, fmasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, 4-irinotecan, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance genie inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfmosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene dichloride, topotecan, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin stimalamer.

The herein provide composition can further comprise one or more additional radiosensitizers. Examples of known radiosensitizers include gemcitabine, 5-fluorouracil, pentoxifylline, and vinorelbine. (Zhang et al., 1998; Lawrence et al., 2001; Robinson and Shewach, 2001; Strunz et al., 2002; Collis et al., 2003; Zhang et al., 2004).

In other aspects, the provided composition(s) can further comprise one or more of classes of antibiotics (e.g., Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, Vancomycin), steroids (e.g., Andranes (e.g., Testosterone), Cholestanes (e.g., Cholesterol), Cholic acids (e.g., Cholic acid), Corticosteroids (e.g., Dexamethasone), Estraenes (e.g., Estradiol), Pregnanes (e.g., Progesterone), narcotic and non-narcotic analgesics (e.g., Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenac Sodium, Diflorasone Diacetate, Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Momiflumate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolic Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tolmetin, Tolmetin Sodium, Triclonide, Triflumidate, Zidometacin, Zomepirac Sodium), or anti-histaminic agents (e.g., Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

Disclosed herein is a patient-based quantitative proteomic study in which the primary goal was to identify molecular correlates of lymph node status in CRC. A secondary goal was to identify markers that distinguish cancer overall from patient-matched, histologically normal colonic epithelium. The study used microdissected specimens from 24 CRC patients stratified by node status. Microdissection enriches for tumor cells and excludes stroma and necrotic tissue, increasing the specificity of the screen.

As disclosed herein, the top-ranked biomarker of node status was transgelin, a 23-kDa actin-binding protein. Paradoxically, there is previous evidence that transgelin is both a tumor suppressor and a variable tumor biomarker, depending on the tumor type, stage, and experimental model [Yang Z, et al. 2007; Nair R R, et al. 2006; Shields J M, et al. 2002; Sitek B, et al. 2005; Qi Y, et al. 2005; Huang Q, et al. 2008; Ryu J W, et al. 2003; Mikuriya K, et al. 2007; Li N, et al. 2007]. As disclosed herein, elevated transgelin levels were predictive of node status, although it did not differ significantly between normal colonic epithelium and cancer overall. Follow-up experiments were also performed with transgelin to investigate its involvement in biologic processes that are known to be relevant to metastatic behavior.

Transgelin, also known as smooth muscle protein 22α (SM22α), is a 201-amino acid protein that contains a calponin homology domain. It is an early marker of smooth muscle differentiation and is also present in the cytoplasm of fibroblasts and some epithelial cells [Lawson D, et al. 1997; Assinder S J, et al. 2009]. Transgelin promotes actin gelling in vitro (the name derives from transformation-sensitive actin gelling protein) [Shapland C, et al. 1993] and is involved in podosome formation in smooth muscle cells, thus predisposing the cells toward migration and invasion [Gimona M, et al. 2003]. It is associated with Ca²⁺-independent vascular contractility [Je H D and Sohn U D. 2007] and is also a direct target of transforming growth factor β (TGF-β)/Smad3-dependent epithelial cell migration in idiopathic pulmonary fibrosis [Yu H, et al. 2008]. These findings are consistent with a physiological role of transgelin in controlling cell motility.

CRC metastasis is a multihit, multistage process [Chiang A C and Massague J. 2008]. In addition to greater motility, cells must be able to invade the extracellular matrix, survive at low density outside the tumor microenvironment, and develop resistance to anoikis, which is a form of apoptosis triggered by loss of cell-matrix interaction. Another process that is frequently associated with metastasis is the epithelial-to-mesenchymal transition (EMT) [Tse J C and Kalluri R. 2007; Greengauz-Roberts O, et al. 2005]. EMT is a transcriptional program that occurs normally during embryonic development and is characterized by changes in expression levels for E-cadherin, a mediator of cell-cell adhesion, and other markers characteristic of mesenchymal and epithelial cells. In the disclosed study, it was investigated whether experimental manipulation of transgelin expression in established CRC cell lines influenced invasion, survival, resistance to anoikis, and the EMT.

i. Results

a. Proteomic Profiling of CRC and Matched Normal Epithelium

CRC samples were collected from 12 node-positive and 12 nodenegative patients (Table 1). Each CRC sample was paired with a sample of uninvolved colonic epithelium obtained from the same patient at the time of resection. Proteomic analysis was performed by LCM of frozen histologic sections, followed by two-dimensional difference gel electrophoresis (2D-DIGE) with an internal standard design [Greengauz-Roberts O, et al. 2005]. A pilot study was performed to evaluate the technical variation based on repeated LCM sampling of the same specimens. The median coefficient of variation (CV) was 9.4% for CRC and 10.1% for normal colonic epithelium. An improvement more than the 23% CVs in a previous report can reflect both differences in the tissue type and the use of a microdissected rather than bulk-dissected internal standard [Arnouk H. 2009]. Assuming a total CV of 50% (dominated by biologic variation because technical variation is only approximately 10%), the group sizes used in the current study provided at least 80% power to identify features with a two-fold change between experimental groups using a two-sided a of 0.05 [Arnouk H. 2009; Molloy M P, et al. 2003].

TABLE 1
Demographics of the Patients Included in the Proteomic Study.

| | Node-Negative | Node-Positive | |
|---|---|---|---|
| | (n = 12) | (n = 12) | P |
| Age (years) | 68.17 ± 11.71 | 62.08 ± 12.30 | .670 |
| Sex | | | |
| Male | 8 | 6 | .680 |
| Female | 4 | 6 | |
| Ethnicity* | | | |
| White | 7 | 6 | .415 |
| African-American | 3 | 6 | |
| Maximum diameter | 4.75 ± 1.99 | 5.32 ± 1.99 | .464 |
| of the tumor (cm) | | | |
| Histologic grade | | | |
| Moderate | 11 | 10 | 1.000 |
| Poor | 1 | 2 | |
| Primary site | | | |
| Left colon | 7 | 6 | 1.000 |
| Right colon | 3 | 4 | |
| Rectum | 2 | 2 | |
| Primary tumor | | | |
| T2 | 2 | 1 | 1.000 |
| T3 | 10 | 10 | |
| T4 | 0 | 1 | |

*The ethnicity information of two patients from the node-negative group was missing.

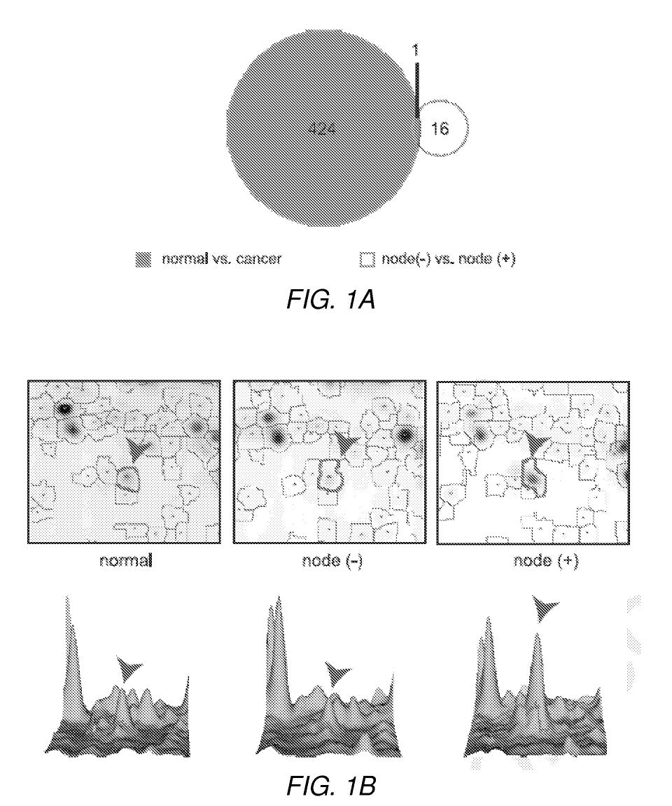

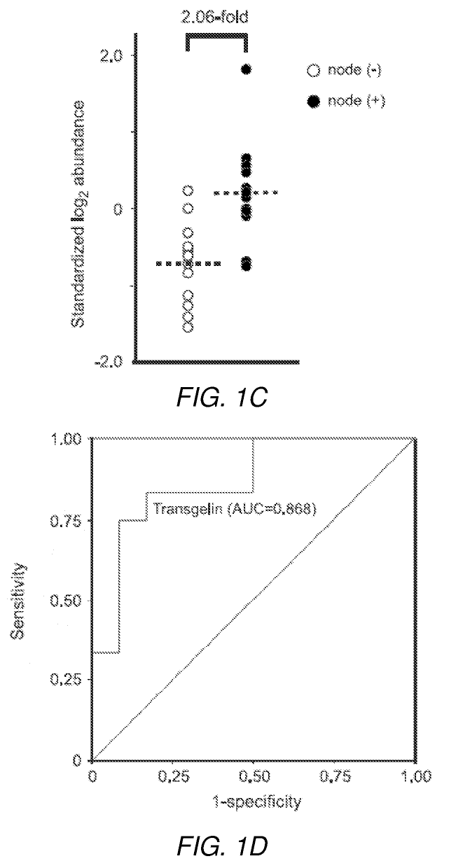

LCM and 2D-DIGE were performed on all 48 samples, and relative abundance values were derived for 980 protein spots that were matched with high confidence across more than 90% of the gels. Significance analysis of microarrays was used to evaluate each intergroup difference and to produce a list of spots, rank-ordered by difference score (D, calculated based on the average difference between groups divided by the sum of the spot-specific scatter [variance] and a measure of scatter [variance] common to all proteins [Tusher V G, et al. 2001].) Applying a liberal 10% false discovery rate cutoff, there were 16 spots that differentiated node-positive from node-negative CRC and 424 spots that differentiated CRC from patient-matched normal colonic epithelium (FIG. 1A). Interestingly, there was no more overlap between these sets than would be predicted by chance. That is, most features that distinguished one group of CRC from another were not useful for differentiating CRC from normal and vice versa.

b. Identification of Proteins by MS

Six protein spots from the node-positive versus node-negative CRC comparison were identified molecularly by MS. Three were metabolic enzymes, and three were isoforms of the actin-binding protein, transgelin (Table 2). On average, the change in transgelin expression levels exceeded two-fold (FIGS. 1, B and C), which was the cutoff level for useful biomarkers that we had assumed in our power calculation. Perhaps more importantly, the area under the receiver operating characteristic curve for a test of node status based on transgelin was 0.868 (P=0.002; FIG. 1D), which is considered excellent for a single marker. Transgelin was selected for further characterization because of its statistical ranking, ability to distinguish between groups, and previous work suggesting a role in control of cell motility [Shapland C, et al. 1993; Gimona M, et al. 2003; Je H D and Sohn U D. 2007; Yu H, et al. 2008].

56 protein spots were also identified that differed in CRC versus patient-matched colonic mucosa (Table 2). Thus, disclosed herein is the use of each of these proteins, alone or in combination with transgelin and/or each other, for use as a biomarker in the disclosed compositions and methods.

TABLE 2
Proteins Identified by 2D-DIGE and MS.

| Accession No. | Protein Name |
|---|---|
| Q01995 | Transgelin |
| P40925 | Cytosolic malate dehydrogenase |
| Q01995 | Transgelin |
| Q01995 | Transgelin |
| P30837 | Aldehyde dehydrogenase X, mitochondrial precursor |
| P49748 | Very long-chain specific acyl-CoA dehydrogenase, mitochondrial precursor |
| P40121 | Macrophage-capping protein |
| Q9Y427 | Tropomyosin 1 α chain isoform 2 |
| P22626 | Heterogeneous nuclear ribonucleoproteins A2/B1 |
| Q8IUD2 | ELKS/RAB6-interacting/CAST family member 1 |
| Q9NZL3 | Zinc finger protein 224 |
| P38919 | Eukaryotic initiation factor 4A-III |
| Q15084 | Protein disulfide-isomerase A6 precursor |
| P67936 | Tropomyosin α-4 chain, isoform 2 |
| P02792 | Ferritin light chain |
| P06702 | Protein S100-A9 |
| P14618 | Pyruvate kinase isozymes M1/M2 |
| Q6ZWB8 | cDNA FLJ41346 fis, clone BRAWH2005315, moderately similar to Neuronal- |
| | STOP protein (MAP6 protein) |
| P10809 | 60 kDa heat shock protein, mitochondrial precursor (HSP60) |
| P08670 | Vimentin |
| P13796 | Plastin-2 |
| P14618 | Pyruvate kinase isozymes M1/M2 |
| P29401 | TKT protein |
| P37837 | Transaldolase 1 |
| O00299 | Chloride intracellular channel protein 1 |
| Q15019 | Septin-2 |
| O00299 | Chloride intracellular channel protein 1 |
| Q7Z5Z4 | SHUJUN-1 |
| Q7KZF4 | Staphylococcal nuclease domain-containing protein 1 (p100 coactivator) |
| O43707 | α-Actinin-4 |
| P00915 | Carbonic anhydrase I |
| P56470 | Galectin 4 |
| P05787 | Keratin, type II cytoskeletal 8 |
| P05787 | Keratin, type II cytoskeletal 8 |
| Q14376 | UDP-galactose-4-epimerase |
| P35900 | Keratin, type I cytoskeletal 20 |
| P17931 | LEG3_HUMAN Galectin-3 (Galactose-specific lectin 3) (Mac-2 antigen) (IgE- |
| | binding) |
| O95994 | Anterior gradient protein 2 homolog precursor |
| O95994 | Anterior gradient protein 2 homolog precursor |
| P05787 | Keratin, type II cytoskeletal 8 |
| P17931 | Galectin-3 |
| O75947 | ATP synthase, H+ transporting, mitochondrial F0 complex, subunit d isoform |
| P13645 | Keratin, type I cytoskeletal 10 |
| P05787 | Keratin, type II cytoskeletal 8 |
| P21796 | Voltage-dependent anion-selective channel protein 1 |
| P62826 | GTP-binding nuclear protein Ran |
| P47985 | Cytochrome b-c1 complex subunit Rieske, mitochondrial precursor (ubiquinol- |
| | cytochrome c reductase iron-sulfur subunit, mitochondrial precursor) |
| Q99798 | Aconitate hydratase, mitochondrial precursor |
| Q8TF65 | PDZ domain protein GIPC2 |
| P52597 F | Heterogeneous nuclear ribonucleoprotein |
| P68871 | Hemoglobin subunit β |
| P30041 | Peroxiredoxin-6 |
| P04075 | Fructose-bisphosphate aldolase A |
| P30048 | Thioredoxin-dependent peroxide reductase, mitochondrial precursor |
| Q9UJ70 N- | Acetylglucosamine kinase |
| A4D0V4 | Capping protein (actin filament) muscle Z-line, α 2 |
| O00151 | PDZ and LIM domain 1 (elfin) |
| P32119 | Peroxiredoxin-2 |
| P00491 | Purine nucleoside phosphorylase |
| Q9H2G3 | CTCL tumor antigen se20-7 [fragment] |
| P00558 | Phosphoglycerate kinase 1 |
| P50213 | Isocitrate dehydrogenase [NAD] subunit, mitochondrial precursor |

| | |
|---|---|
| MASCOT Algorithm | SEQUEST Algorithm |

| Accession No. | MASCOT Score | CI (%) | P | Sf Score |
|---|---|---|---|---|
| Q01995 | 96 | 100 | | |
| P40925 | | | 2.584e−08 | 3.587517 |
| Q01995 | 127 | 100 | | |
| Q01995 | 145 | 100 | | |
| P30837 | 72 | 99.102 | | |
| P49748 | 108 | 100 | | |
| P40121 | 68 | 98.079 | | |
| Q9Y427 | | | 3.982e−09 | 2.785449 |
| P22626 | 181 | 100 | | |
| Q8IUD2 | 73 | 99.406 | | |
| Q9NZL3 | 68 | 98.166 | | |
| P38919 | 120 | 100 | | |
| Q15084 | 122 | 100 | | |
| P67936 | 115 | 100 | | |
| P02792 | 145 | 100 | | |
| P06702 | 74 | 99.433 | | |
| P14618 | 146 | 100 | | |
| Q6ZWB8 | 75 | 99.55 | | |
| P10809 | 126 | 100 | | |
| P08670 | 184 | 100 | | |
| P13796 | 113 | 100 | | |
| P14618 | 174 | 100 | | |
| P29401 | 66 | 96.813 | | |
| P37837 | | | .0001081 | 1.946282 |
| O00299 | | | 1.284e−08 | 1.903239 |
| Q15019 | | | 2.973e−09 | 5.429678 |
| O00299 | | | 4.272e−10 | 1.872898 |
| Q7Z5Z4 | 105 | 100 | | |
| Q7KZF4 | 76 | 99.681 | | |
| O43707 | 170 | 100 | | |
| P00915 | | | 1.535e−11 | 10.67389 |
| P56470 | | | 7.614e−10 | 3.753924 |
| P05787 | 281 | 100 | | |
| P05787 | 315 | 100 | | |
| Q14376 | | | 7.494e−10 | 4.446128 |
| P35900 | 88 | 99.978 | | |
| P17931 | | | 2.435e−09 | 1.997028 |
| O95994 | 85 | 99.962 | | |
| O95994 | 78 | 99.799 | | |
| P05787 | 260 | 100 | | |
| P17931 | 110 | 100 | | |
| O75947 | | | 5.883e−06 | 0.9162297 |
| P13645 | 70 | 98.64 | | |
| P05787 | 181 | 100 | | |
| P21796 | 159 | 100 | | |
| P62826 | 84 | 99.953 | | |
| P47985 | 103 | 100 | | |
| Q99798 | 95 | 99.996 | | |
| Q8TF65 | | | 1.683e−10 | 1.945089 |
| P52597 F | 109 | 100 | | |
| P68871 | 133 | 100 | | |
| P30041 | 82 | 99.91 | | |
| P04075 | 95 | 99.996 | | |
| P30048 | 67 | 97.224 | | |
| Q9UJ70 N- | 71 | 98.945 | | |
| A4D0V4 | | | 1.175e−06 | 1.73486 |
| O00151 | | | 5.059e−09 | 1.168792 |
| P32119 | 112 | 100 | | |
| P00491 | 130 | 100 | | |
| Q9H2G3 | 65 | 95.498 | | |
| P00558 | 67 | 97.287 | | |
| P50213 | 76 | 99.689 | | |

| | | Sequence | | | | | |
|---|---|---|---|---|---|---|---|
| Accession No. | Peptides | Coverage (%) | MW | pI | DScore | Fold Change | FDR (%) |
| Q01995 | 11 | 44 | 22,653 | 8.87 | 2.85 | 2.06 | 0 |
| P40925 | 4 | 18 | 36,403 | 7.17 | 2.33 | 1.52 | 0 |
| Q01995 | 11 | 46 | 22,653 | 8.87 | 2.14 | 1.89 | 7.18 |
| Q01995 | 15 | 61 | 22,653 | 8.87 | 1.99 | 2.04 | 7.18 |
| P30837 | 14 | 29 | 57,658 | 6.36 | −2.65 | −1.96 | 0 |
| P49748 | 17 | 21 | 70,745 | 8.92 | −2.13 | −1.75 | 0 |
| P40121 | 5 | 13 | 38,786 | 5.32 | 3.31 | 2.55 | 0 |
| Q9Y427 | 3 | 13.03 | 32,658 | 4.25 | 2.93 | 2.26 | 0 |
| P22626 | 11 | 49 | 37,464 | 8.97 | 2.87 | 1.71 | 0 |
| Q8IUD2 | 33 | 21 | 108,840 | 6.19 | 2.83 | 1.80 | 0 |
| Q9NZL3 | 25 | 27 | 84,894 | 9.03 | 2.57 | 1.65 | 0 |
| P38919 | 16 | 36 | 47,088 | 6.08 | 2.5 | 1.81 | 0 |
| Q15084 | 14 | 32 | 46,512 | 4.95 | 2.39 | 1.68 | 0 |
| P67936 | 12 | 34 | 32,874 | 4.69 | 2.32 | 1.90 | 0 |
| P02792 | 6 | 44 | 16,441 | 5.65 | 2.31 | 2.16 | 0 |
| P06702 | 7 | 77 | 13,291 | 5.71 | 2.24 | 2.17 | 0 |
| P14618 | 20 | 39 | 58,470 | 7.96 | 2.22 | 1.62 | 0 |
| Q6ZWB8 | 20 | 32 | 51,297 | 8.88 | 2.22 | 1.61 | 0 |
| P10809 | 17 | 27 | 61,187 | 5.70 | 2.2 | 1.58 | 0 |
| P08670 | 28 | 55 | 53,676 | 5.06 | 2.2 | 1.59 | 0 |
| P13796 | 18 | 26 | 70,815 | 5.20 | 2.14 | 1.50 | 0 |
| P14618 | 17 | 35 | 58,512 | 7.96 | 1.96 | 1.73 | 0 |
| P29401 | 10 | 26 | 50,335 | 8.02 | 1.96 | 1.80 | 0 |
| P37837 | 4 | 13.35 | 37,516 | 6.00 | 1.94 | 1.47 | 0 |
| O00299 | 2 | 14.29 | 23,528 | 4.25 | 1.94 | 1.66 | 0 |
| Q15019 | 5 | 17.24 | 46,556 | 6.00 | 1.89 | 1.52 | 0 |
| O00299 | 2 | 14.29 | 23,528 | 4.25 | 1.89 | 1.37 | 0 |
| Q7Z5Z4 | 7 | 39 | 17,047 | 4.21 | 1.88 | 1.37 | 0 |
| Q7KZF4 | 26 | 29 | 100,313 | 6.62 | 1.86 | 1.43 | 0 |
| O43707 | 28 | 33 | 102,661 | 5.27 | 1.84 | 1.44 | 0 |
| P00915 | 9 | 56.30 | 28,852 | 7.13 | −6.54 | −4.90 | 0 |
| P56470 | 5 | 19.20 | 35,918 | 10.07 | −5.06 | −3.05 | 0 |
| P05787 | 27 | 48 | 55,874 | 5.62 | −4.33 | −3.41 | 0 |
| P05787 | 26 | 50 | 53,641 | 5.52 | −3.99 | −2.67 | 0 |
| Q14376 | 4 | 18.39 | 38,257 | 6.00 | −3.84 | −2.36 | 0 |
| P35900 | 19 | 41 | 48,514 | 5.52 | −3.68 | −2.28 | 0 |
| P17931 | 6 | 28.40 | 26,172 | 9.88 | −3.65 | −2.18 | 0 |
| O95994 | 7 | 42 | 20,024 | 9.03 | −3.43 | −2.78 | 0 |
| O95994 | 7 | 42 | 20,024 | 9.03 | −3.36 | −2.76 | 0 |
| P05787 | 25 | 46 | 55,787 | 5.62 | −3.12 | −2.50 | 0 |
| P17931 | 5 | 46 | 15,630 | 9.41 | −3.08 | −1.95 | 0 |
| O75947 | 2 | 8.07 | 18,480 | 4.25 | −2.89 | −1.84 | 0 |
| P13645 | 19 | 31 | 59,020 | 5.09 | −2.84 | −1.74 | 0 |
| P05787 | 19 | 35 | 53,641 | 5.52 | −2.7 | −1.94 | 0 |
| P21796 | 11 | 43 | 30,818 | 8.63 | −2.55 | −1.78 | 0 |
| P62826 | 4 | 21 | 23,307 | 9.02 | −2.33 | −1.65 | 0 |
| P47985 | 8 | 30 | 29,959 | 8.55 | −2.31 | −1.67 | 0 |
| Q99798 | 20 | 29 | 86,113 | 7.36 | −2.31 | −1.61 | 0 |
| Q8TF65 | 2 | 7.62 | 34,333 | 6.00 | −2.14 | −1.56 | 0 |
| P52597 F | 8 | 23 | 45,985 | 5.38 | −1.99 | −1.50 | 0 |
| P68871 | 8 | 54 | 15,970 | 7.98 | −1.96 | −1.64 | 0 |
| P30041 | 12 | 46 | 25,133 | 6.00 | −1.92 | −1.55 | 0 |
| P04075 | 13 | 35 | 39,852 | 8.30 | −1.89 | −1.76 | 0 |
| P30048 | 6 | 26 | 26,107 | 7.04 | −1.84 | −1.55 | 0 |
| Q9UJ70 N- | 11 | 35 | 37,694 | 5.81 | −1.84 | −1.51 | 0 |
| A4D0V4 | 3 | 17.83 | 32,929 | 5.13 | −1.81 | −1.43 | 0 |
| O00151 | 2 | 9.42 | 36,049 | 6.00 | −1.8 | −1.44 | 0 |
| P32119 | 10 | 38 | 22,014 | 6.84 | −1.8 | −1.49 | 0 |
| P00491 | 11 | 43 | 32,325 | 6.45 | −1.78 | −1.60 | 0 |
| Q9H2G3 | 25 | 24 | 73,884 | 5.57 | −1.77 | −1.59 | 0 |
| P00558 | 12 | 29 | 44,973 | 8.30 | −1.73 | −1.49 | 0 |
| P50213 | 9 | 28 | 40,022 | 6.47 | −1.72 | −1.40 | 0 |

c. Transgelin Expression Patterns on TMAs

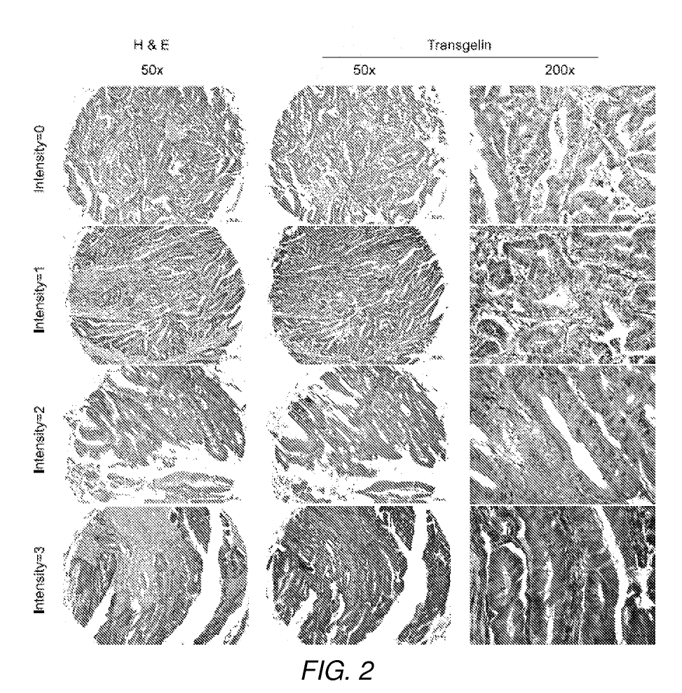

The correlation between transgelin expression and node status was further investigated in a larger, independent patient cohort by immunohistochemical staining of commercial TMAs. TMAs from two sources were analyzed, representing 94 eligible cases from different geographic regions. Representative patterns of transgelin staining are shown in FIG. 2. Staining was evident in the cytoplasm and in some cell nuclei. Two independent investigators scored the TMAs blindly, using a 0 to 9 scale representing a combination of staining intensity and fraction of cells stained (see Materials and Methods [Wang Z, et al. 2008]). Increased frequency of moderate- and high-level transgelin expression in node-positive CRC was seen in this cohort. The distribution of scores for the node-negative and node-positive groups was significantly different, based on aWilcoxon rank sum test (P=0.036; Table 3).

TABLE 3
Summary of transgelin expression on tissue microarrays

| | Node-negative | Node-positive | |
|---|---|---|---|
| Staining (score) | CRC (n) | CRC (n) | Total (n) |
| None (0) | 30 (62.5%) | 20 (43.5%) | 50 |
| Low (1-3) | 12 (25%) | 12 (26.1%) | 24 |
| Moderate (4-6) | 4 (8.3%) | 11 (23.9%) | 15 |
| High (7-9) | 2 (4.2%) | 3 (6.5%) | 5 |
| Total (n) | 48 | 46 | 94 |

| | |
|---|---|
| P value | 0.036 |

One limitation of TMAs is that each sample represents only a very small region of the tumor (1.0- to 1.5-mm tissue core). Because of this, a clonal subpopulation of cells that express a metastatic marker might easily be missed, resulting in a type II error. Nevertheless, the statistically significant association between transgelin and node status supports the findings from quantitative proteomic analysis.

d. Establishment of a Cell Culture Model

To investigate the potential mechanisms by which transgelin might promote metastasis in CRC, it was desirable to establish an in vitro cell culture model. A number of CRC cell lines were screened for transgelin expression, and HCT116 and SW480 were selected for studies based on their moderate levels of endogenous transgelin expression and the ease with which expression of the transgelin gene (gene symbol: TAGLN) could be manipulated genetically.

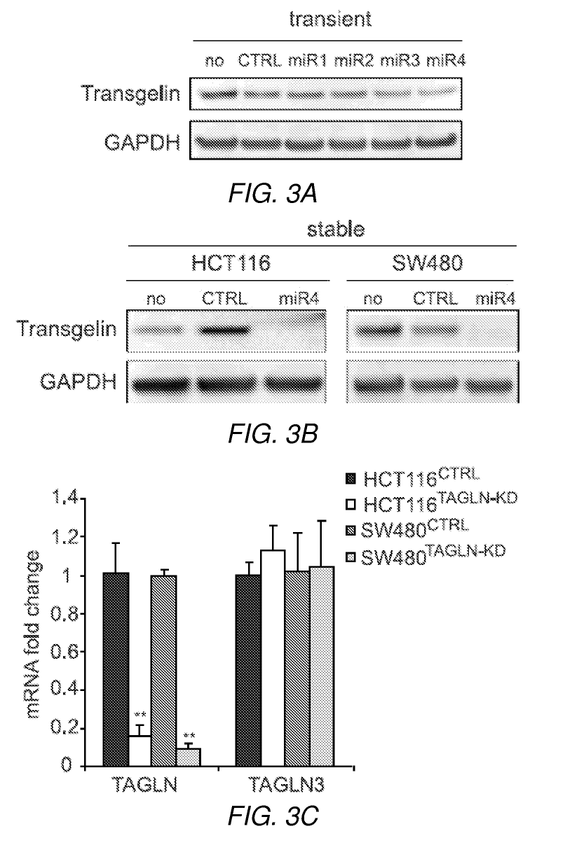

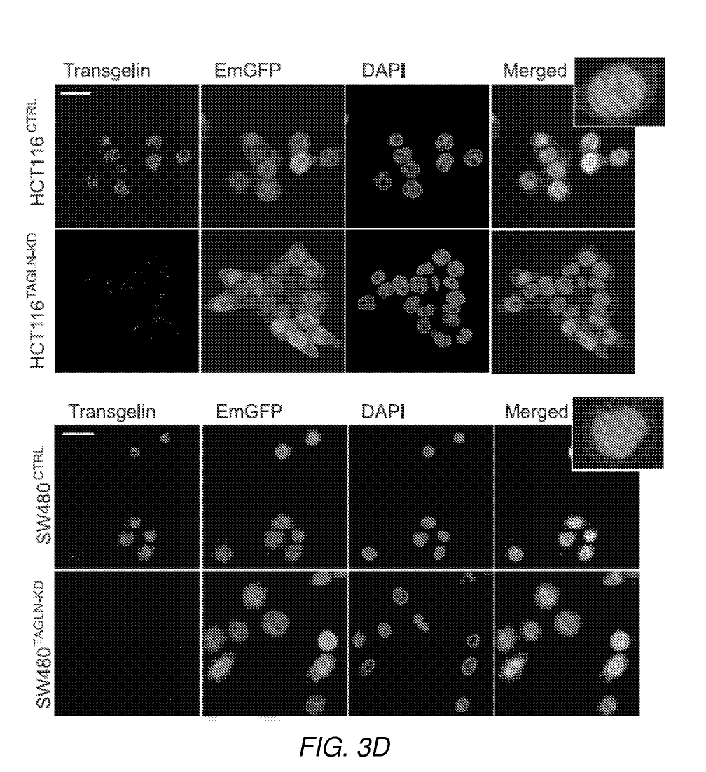

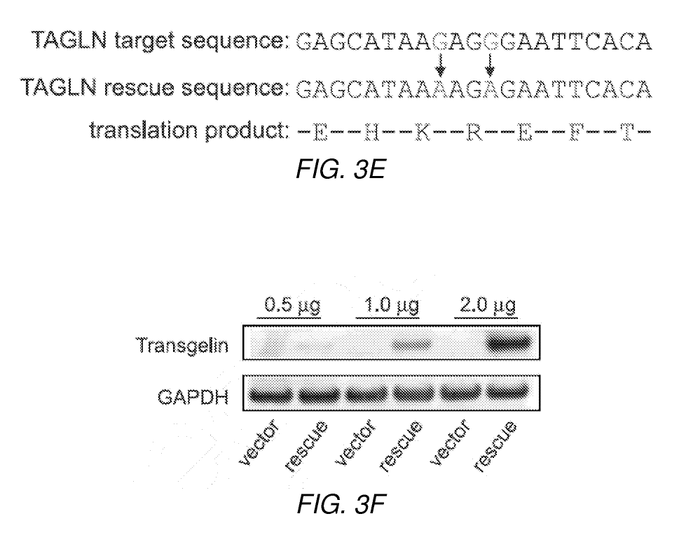

Four different miRNA sequences were tested for their ability to silence transgelin in HCT116 cells (FIG. 3A) and used the most effective of these, TAGLN miRNA-4, to establish stably transfected populations. These populations (HCT116^{TAGLN-KD}, SW480^{TAGLN-KD}) and the matched control miRNA-transfected populations (HCT116^{CTRL}, SW480^{CTRL}) were characterized for transgelin protein expression by immunoblot analysis (FIG. 3B), for TAGLN mRNA by real-time RT-PCR (FIG. 3C), and immunofluorescence staining with antitransgelin antibody (FIG. 3D). A greater than 80% decrease in expression was observed in all three assays. Expression of the closely related TAGLN3 mRNA was not significantly altered, confirming the specificity of the TAGLN miRNA-4 (FIG. 3C). To exclude more rigorously off-target effects in subsequent experiments, a TAGLN rescue plasmid was also created that expressed a TAGLN miRNA-4-resistant cDNA (FIG. 3E). Transient transfection with this cDNA restored transgelin expression in the HCT116^{TAGLN-KD} population (FIG. 3F).

e. Effects of Transgelin on Cell Invasion, Survival, and Anoikis

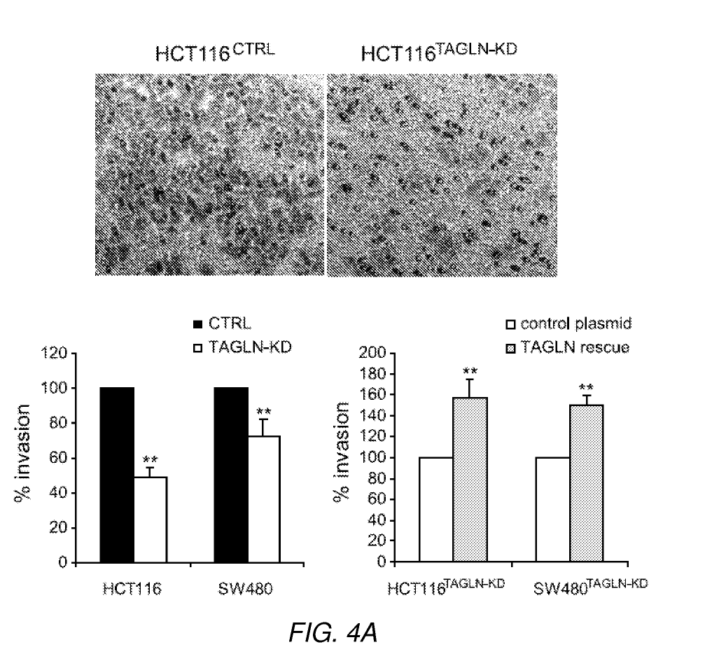

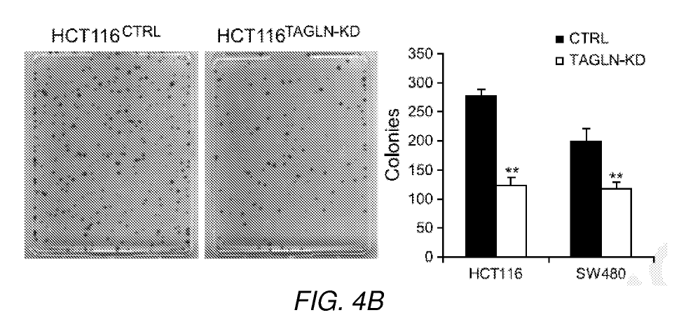

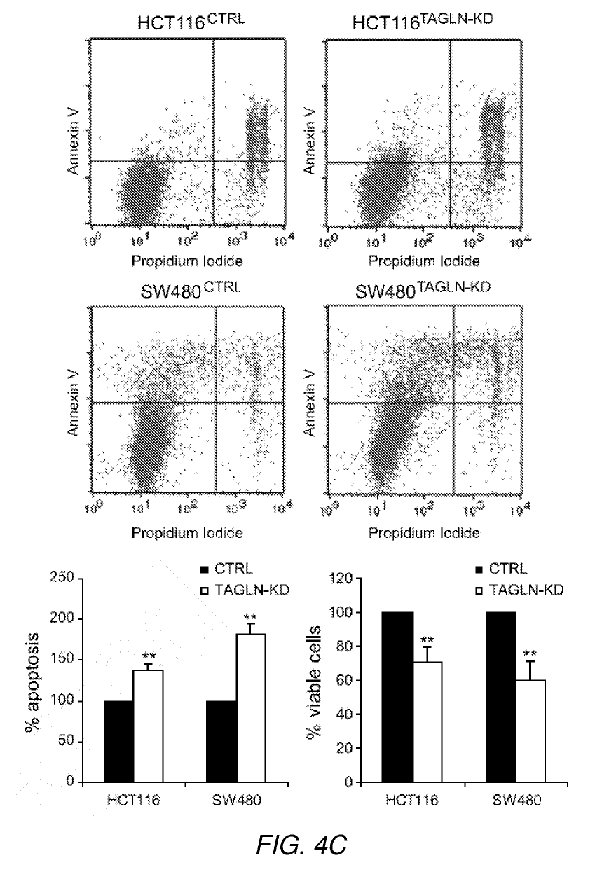

To evaluate the effect of altered transgelin expression level on biologic processes that are relevant to metastasis, in vitro assays were performed for invasion, survival at low density, and anoikis. Invasion was measured using a Transwell assay. Cells were seeded in serum-free medium in an upper chamber, which is separated by a Matrigel-coated filter from a lower chamber that contains medium with 10% fetal bovine serum as a chemoattractant. Results were measured based on staining of cells that invaded the Matrigel, migrated through pores in the filter, and reached the lower surface. Knockdown of transgelin reduced invasion by more than 50% in HCT116 cells and by 27%in SW480 cells (P<0.01; FIG. 4A). Transient transfection with TAGLN rescue plasmid, but not control plasmid, significantly restored invasion capability to both HCT116 and SW480 TAGLN knockdown cells (FIG. 4A). On the basis of the successful rescue by cDNA transfection, it is unlikely that results are attributable to off-target effects of the miRNA.

Clonogenic survival was evaluated by plating cells at low density and scoring for colony formation after 10 to 14 days. Knockdown of transgelin reduced the clonogenic survival of HCT116 cells by approximately 55% and SW480 cell by approximately 40% (P<0.01; FIG. 4B).

Resistance to anoikis (apoptosis induced by loss of cell-matrix contact) was investigated by plating control and knockdown cells on polyHEMA-coated dishes. After 72 hours, cells were characterized by flow cytometry using Annexin V and propidium iodide stains (FIG. 4C). The total fraction of apoptotic cells in the TAGLN knockdown groups, based on Annexin V-positive staining, increased by 1.4- to 1.8-fold, relative to the corresponding control cells (P<0.01). The percentage of viable cells in the same population was measured independently by replating on regular dishes, incubating for 24 hours, removing the unattached cells, and counting the remainder. Knockdown of transgelin reduced the percentage of viable cells to 60% to 70% of control values (P<0.01; FIG. 4C). Together, these results strongly implicate transgelin in resistance to anoikis.

f. Effects of Transgelin Expression on Epithelial and Mesenchymal Markers

Transgelin is normally expressed in mesenchymal cells, and its appearance in tumor cells of epithelial origin is consistent with the engagement of the EMT, by which tumor cells acquire a more aggressive phenotype. Transgelin is localized primarily in the nucleus of CRC cells (FIG. 3D) and an 85% similar homolog, transgelin 3, is a transcription factor that controls expression of the gonadotropin gene [Feng J, et al. 2008]. Thus, whether transgelin is a transcriptional regulator was evaluated.

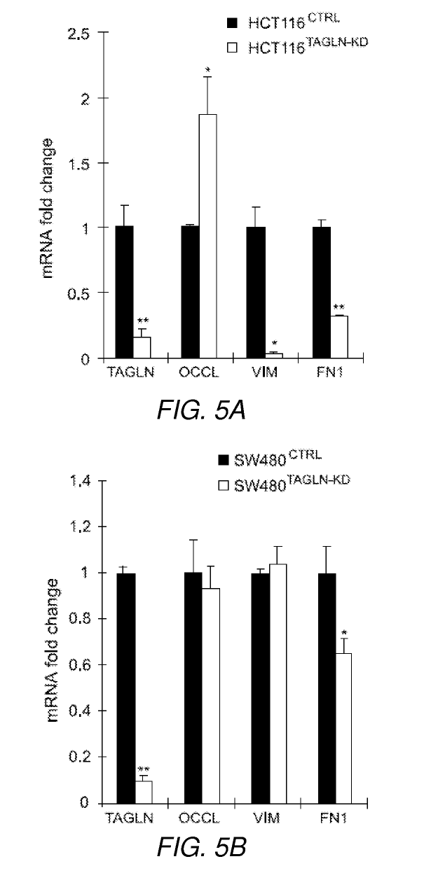

During the EMT, the cell intermediate filament system switches from a keratin-rich network that connects to adherens junctions and hemidesmosomes to a vimentin-rich network that connects to focal adhesions [Kokkinos M I, et al. 2007]. Typical elements of the EMT include loss of expression of proteins associated with adherens junctions (such as cadherins and catenins) and tight junctions (such as occludin) and an increase in expression of mesenchymal intermediate filament protein, vimentin, and the mesenchymal adhesion protein, fibronectin [Yu H, et al. 2008; Kokkinos M I, et al. 2007; Kaplan R N, et al. 2005; Hugo H, et al. 2007], in control and transgelin knockdown cells. In HCT116 cells, knockdown of transgelin was associated with the up-regulation of mRNA encoding occludin and with the down-regulation of mRNA encoding fibronectin-1 and the mesenchymal intermediate filament protein vimentin (FIG. 5A). The mRNA levels for two other epithelial markers, E-cadherin and β-catenin, were unaffected. In SW480 cells, transgelin knockdown had less effect. Of the mRNA investigated, only fibronectin-1 was significantly influenced (FIG. 5B). The finding that alteration of transgelin affects mRNA levels for some, but not all, EMT-associated genes supports a conclusion that transgelin is a promoter-selective regulator of transcription. An ability to regulate transcription of other genes provides a mechanism by which transgelin might broadly influence processes relevant to metastasis.

g. Manipulation of Transgelin Levels Causes a Dramatic Change in the Pattern of Tumor Spread in a Xenograft Model.

Although some clues to metastatic potential can be obtained in tissue culture, the host microenvironment is essential for completion of the process, and this can only be monitored in vivo. Several mouse xenograft models have been used for studies of CRC metastasis, including orthotopic implantation, intrasplenic or portal vein injection, and tail vein injection. For exploratory experiments, the tail vein model was chosen, primarily because it is rapid, does not require special expertise, and can be performed with minimal pain and distress (i.e., without surgical procedures).

The experimental design used 10 animals per group. HCT116 control and TAGLN knockdown cells (2×10⁶) were suspended in 0.15 ml of Hank's solution, filtered to obtain a single-cell suspension, and injected using a 28-gauge needle into a tail vein of six-week-old CB.17 scid mice. Mice were sacrificed after 6-8 weeks or when they exhibited signs of disease (weight loss and decreased grooming). Lungs and other organs that showed gross tumors visible at necropsy were fixed, paraffin embedded, and stained with hematoxylin and eosin for pathological examination. Tumors in lung, heart, kidney, rib, back (near the injection site), leg, and foot were scored and diagnosis was confirmed by a pathologist.

It was investigated whether there was a decrease in the incidence of lung tumors in animals receiving TAGLN knockdown versus control cells. There was in fact a difference of more than two-fold: 33% incidence of lung tumors in the knockdown group (excluding one mouse that died at the time of injection) versus 70% incidence in the control group.

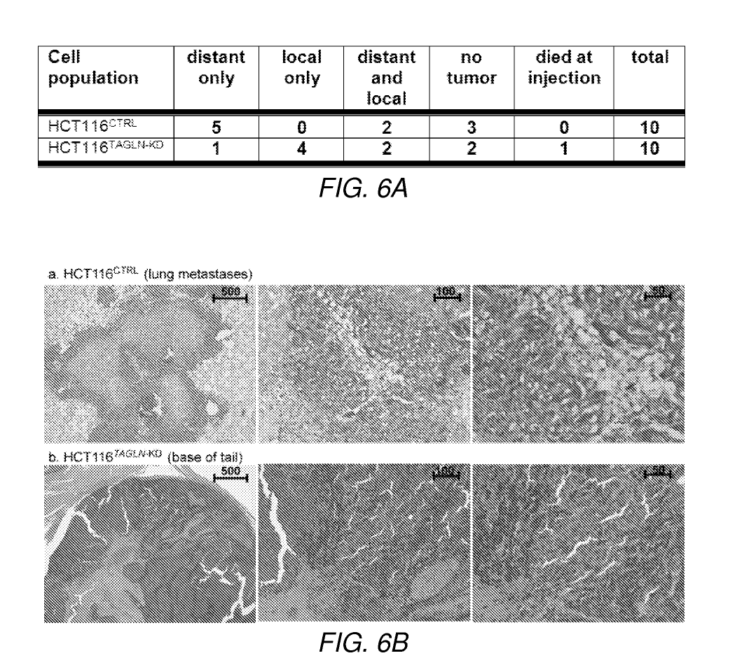

The experiment provided an additional finding. A number of mice in the control group developed tumors near the site of injection, on the back near the base of the tail. The data was reanalyzed taking into account this observation. In FIG. 6A, the pattern of spread was classified in each animal as “local only” (back, leg, and hind foot) “distant” (lung, heart, rib, kidney), both, or neither (no tumor). There was a marked difference between the two experimental groups. In the TAGLN knockdown group, 4/9 mice showed the “local only” pattern, vs. 0/10 in the control group. Conversely only 1/9 mice showed the “distant” pattern, vs 5/10 in the control group. This difference is significant (P=0.03, Fisher's exact test, two-tailed). The histology of the tumors, shown in FIG. 6B, was determined by a pathologist to be similar. Only the pattern of spread differed, which supporting the conclusion that transgelin is important in determining metastatic behavior, rather than tumorigenicity per se.

h. Transgelin Connects with Known Pathways Controlling Cell Growth and Invasiveness.

Although nearly 1000 proteins were quantified in the initial proteomic survey, this represents only a small sampling of the human proteome. Many proteins go undetected because they are too rare, not well resolved in the 2-DE system used, or obscured by other protein species. Bioinformatic analysis can help “fill in the gaps” by suggesting how observed proteins are connected to each other or to known regulatory pathways. The STRING tool (string.db.org) was first used, which draws on databases, text mining, homology, and other sources of information to map connectivity within the proteome [Jensen L J, et al. 2009]. Results (FIG. 7) indicate many lines of connection between the TAGLN gene product and the TGF-β/SMAD regulatory network. Indeed, three of the genes shown in the figure (TGFBR1, SMAD4, and SMAD3) are among those commonly mutated in CRC. JUN and MYC are growth-promoting genes connected not only with the TGF-β/SMAD pathway (FIG. 7, Table 4) but also with RTK and PI3K oncogenic pathways. KLF4 is a CRC tumor suppressor and a target of p53 signaling [Wei D, et al. 2006]. ACTG2, MYOCD, TPM1 (encoding actin, myosin, and tropomyosin, respectively) and related genes are involved in cell motility. Multiple connections between transgelin and known pathways controlling cell growth and invasiveness support the conclustion that that transgelin is a novel determinant of metastatic potential in human CRC.

i. A Recursive Proteomic Analysis Confirms and Extends Findings from Other Approaches and Provides Additional Candidate Biomarkers.

In the initial proteomic analysis, clinical samples were stratified by node status. It was reasoned that it could be fruitful, for exploratory purposes, to restratify the samples according to high or low transgelin level and repeat the SAM analysis. The purpose was to identify proteins that were coregulated with transgelin itself. Restratification affected only 4 of 12 samples: two node-positive samples were reclassified in the low-transgelin group, and two node-negative samples were reclassified in the high-transgelin group. This is referred to as a “recursive” proteomic analysis, in which samples are classified according to the level of a candidate biomarker, rather than the original clinical indicator. Proteins were classified as potentially significant if the SAM false discovery rate was <10% in the new analysis and robust identification by mass spectrometry.

Combining recursive proteomic analysis (an experimental approach) and bioinformatics (a theoretical approach) was successful in identifying several more interesting candidate biomarkers. (FIG. 8, Table 4) Vimentin (VIM) was up-regulated in the high-transgelin group, consistent with the engagement of EMT, as were other cytoskeletal proteins, forming a robust cluster. Interestingly, another cluster, connected robustly to each other and more weakly to the cytoskeletal cluster, contains proteins previously implicated in CRC metastasis, including HSPB1 (HSP27), MAPKAPK2, SRC, RACK1 (which can be involved in either activation or inactivation of SRC, depending on cellular context), and PRKCE, a protein kinase C isoform. The presence of several gene products previously known to be involved in CRC metastasis within this set increases confidence in the methods used. Two metabolic enzymes were up-regulated (FIG. 8) and two were down-regulated (mitochondrial aldehyde dehydrogenase and isocitrate dehydrogenase).

ii. Materials and Methods

a. Tissue Specimens

CRC specimens paired with corresponding normal tissue were obtained from the South Carolina Cancer Tissue Bank, University of South Carolina. Twenty-four patients with CRC (12 node-negative, 12 node-positive), who underwent surgery without presurgical chemotherapy or radiation therapy during 2003 to 2005, were included (Table 1). The institutional review boards of the University of South Carolina and the Medical College of Georgia approved the study, and informed consent was obtained from all patients. Tissues were snap frozen in liquid nitrogen and stored at −80° C. Two independent pathologists confirmed diagnosis of all samples used in the study. Tissue microarrays (TMAs) used in the confirmatory studies represented 94 cases of eligible CRC specimens (48 node-negative, 46 node-positive) and were purchased from US Biomax and Petagen.

b. Laser Capture Microdissection, Two-Dimensional Difference Gel Electrophoresis, and Image Analysis

Laser capture microdissection (LCM) was performed as described [Greengauz-Roberts O, et al. 2005]. Caps with microdissected cells (2500 per sample) were transferred into 90 μl of lysis buffer, incubated at room temperature for 1 hour, sonicated, and centrifuged to remove insoluble material. For each analytical gel, 4 μg of cell lysate was reacted with 2 nmol of Tris (2-carboxyethyl) phosphine hydrochloride (Sigma-Aldrich), then with 4 nmol of CyDye DIGE Fluor, Cy5, for saturation labeling (GE Healthcare Life Sciences). Aliquots of cell lysate from each sample were also pooled to create an internal standard, which was labeled with CyDye DIGE Fluor, Cy3, for saturation labeling. For analytical gels, 8 μg of protein (4 μg of Cy5-labeled sample and 4 μg of Cy3-labeled internal standard) was loaded on a 24-cm pH 3 to 10 nonlinear immobilized pH gradient strip and focused in an IPGphor apparatus (GE Healthcare Life Sciences) for approximately 55,000 volt-hours. Strips were applied to a 12.5% sodium dodecyl sulfate-polyacrylamide gel and electrophoresis was performed at 11 mA per gel overnight at 20° C. in an Ettan DALTtwelve Separation Unit (GE Healthcare Life Sciences). For mass spectrometry (MS), 280 μg of internal standard was reduced with 140 nmol of Tris (2-carboxyethyl) phosphine hydrochloride and labeled with 280 nmol of the Cy3 CyDye before isoelectric focusing and sodium dodecyl sulfate-polyacrylamide gel and electrophoresis [Arnouk H. 2009].

c. Image Acquisition and Analysis

Images were acquired using a Typhoon Trio Imager and protein spots were defined and matched using the DeCyder 6.5 software package (GE Healthcare Life Sciences). Intensity data were exported, log-transformed, and normalized as described [Weinberger P M, et al. 2009]. Candidate biomarkers were identified and ranked using the Significance Analysis of Microarrays (version 3.0, available at http://www-stat.stanford.edu/˜tibs/SAM/).

d. Mass Spectrometry

Protein spots in a preparative gel were matched to a master image of the internal standard from the analytical gels. Spots of interest were excised, digested with trypsin, and extracted [Greengauz-Roberts O, et al. 2005]. Peptides were analyzed by matrix-assisted laser desorption/ionization-MS/MS using the ABI 4700 Proteomics Analyzer (Applied Biosystems) or by liquid chromatography-MS/MS using an LTQ ion trap mass spectrometer (Thermo Scientific). Protein identities were determined using Mascot (available at http://www.matrixscience.com; Matrix Science) or the Sequest algorithm as implemented by the BioWorks Browser v3.2 (Thermo Scientific) and the National Center for Biotechnology Information database. Autodigested trypsin peaks were used as an internal mass calibration standard. Evaluation of significance was based on the score particular to the method used, the sequence coverage, and the consistency between experimental and predicted molecular weight and pl.

e. Cell Lines and Transfection

Human colon carcinoma cell lines HCT116 and SW480 were purchased from the American Type Culture Collection and maintained according to their protocols. MicroRNA (miRNA) plasmids targeting TAGLN were generated using the pcDNA 6.2-GW/EmGFP-miR vector (Invitrogen; refer to Table 4 for inserted sequences). After lipofectamine-mediated transfection, stable transfectants were selected and cultured in medium containing 5 μg/ml blasticidin (Invitrogen). A double-point mutation was introduced into the full-length TAGLN complementary DNA (cDNA; Open Biosystems) using the QuikChange Lightning Site-Directed Mutagenesis Kit (Stratagene) to create a TAGLN rescue cDNA. The rescue TAGLN sequence was transferred into pDONR 221, then pcDNA-DEST40 (Invitrogen) by site-directed recombination. Rescue of TAGLN expression and function was assayed at 48 hours after lipofectamine-mediated cDNA transfection.

TABLE 4
Sequences Inserted in TAGLN miRNA Plasmids.

| DNA Oligos | Top Strand (5′-3′) | SEQ ID |
|---|---|---|
| Hmi416872 | TGCTGATCTGAAGGCCAATGACATGCGTTTTG | SEQ ID |
| top | GCCACTGACTGACGCATGTCAGGCCTTCAGAT | NO: 1 |
| Hmi416872 | CCTGATCTGAAGGCCTGACATGCGTCAGTCAG | SEQ ID |
| bottom | TGGCCAAAACGCATGTCATTGGCCTTCAGATC | NO: 2 |
| Hmi416873 | TGCTGAACTGATGATCTGCCGAGGTCGTTTTG | SEQ ID |
| top | GCCACTGACTGACGACCTCGGGATCATCAGTT | NO: 3 |
| Hmi416873 | CCTGAACTGATGATCCCGAGGTCGTCAGTCAG | SEQ ID |
| bottom | TGGCCAAAACGACCTCGGCAGATCATCAGTTC | NO: 4 |
| Hmi416874 | TGCTGTGCACTTCGCGGCTCATGCCAGTTTTG | SEQ ID |
| top | TCCACTGACTGACTGGCATGACGCGAAGTGCA | NO: 5 |
| Hmi416874 | CCTGTGCACTTCGCGTCATGCCAGTCAGTCAG | SEQ ID |
| bottom | TGGCCAAAACTGGCATGAGCCGCGAAGTGCAC | NO: 6 |
| Hmi416875 | TGCTGTGTGAATTCCCTCTTATGCTCGTTTTG | SEQ ID |
| top | GCCACTGACTGACGAGCATAAGGGAATTCACA | NO: 7 |
| Hmi416875 | CCTGTGTGAATTCCCTTATGCTCGTCAGTCAG | SEQ ID |
| bottom | TGGCCAAAACGAGCATAAGAGGGAATTCACAC | NO: 8 |

f. Immunoblot Analysis, Immunohistochemistry, and Immunofluorescence

Immunoblot analysis was carried out using antitransgelin immunoglobulin (IgG) and anti-GAPDH IgG1 (Abcam) with ECF substrate for detection. Immunohistochemical staining was performed using the Histostain-Plus Kit (DAB, Broad Spectrum; Invitrogen) and assessed blindly by two independent investigators. The staining of transgelin was scored as the product of the staining intensity (on a scale of 0-3: negative=0, weak=1, moderate=2, strong=3) and the percentage of cells stained (on a scale of 0-3: 0=zero, 1=1%-25%, 2=26%-50%, 3=51%-100%), resulting in scores on a scale of 0 to 9 [Wang Z, et al. 2008]. For immunofluorescence, cells were fixed in 4% paraformaldehyde for 15 minutes, permeabilized with 0.1% Triton X-100 for 5 minutes, and incubated with blocking buffer (15% goat serum, 0.2% fish skin gelatin and 0.03% NaN3 in phosphate-buffered saline) for 30 minutes at room temperature. Cells were sequentially incubated with anti-transgelin IgG and anti-rabbit IgG conjugated to Alexa Fluor 594 (Molecular Probes). Slides were mounted using VECTASHIELD 16 mounting medium with DAPI (Vector Laboratories). Images were collected using a meta confocal microscope (Zeiss LSM 510).

g. RNA Isolation and Real-Time Polymerase Chain Reaction

Extraction of total RNA was performed using Trizol (Invitrogen) followed by reverse transcription (RT). Real-time polymerase chain reaction (PCR) was carried out using an MJ PTC-200 Chromo4 thermocycler (Bio-Rad) with a SYBR Green PCR kit (Qiagen). Data analysis was performed using OpticonMonitor software (version 3.1; Bio-Rad). PCR primers are listed in Table 5.

TABLE 5
Primers for Real-time RT-PCR.

| Gene | | SEQ ID |
|---|---|---|
| | Forward Primer (5′-3′) | |
| TAGLN | GTTCCAGACTGTTGACCTCTTT | SEQ ID NO: 9 |
| TAGLN3 | ATGGGAAGGGAAGGACATGGC | SEQ ID NO: 11 |
| ECAD | ATACACTCTCTTCTCTCACGCTGTG | SEQ ID NO: 13 |
| OCOL | CCTGATGAATTCAAACCGAATC | SEQ ID NO: 15 |
| CTTNB1 | AAATGCTTGGTTCACCAGTGGAT | SEQ ID NO: 17 |
| VIM | TTCAGACAGGATGTTGACAATGC | SEQ ID NO: 19 |
| FN1 | CGAGAGTAAACCTGAAGCTGAAGAG | SEQ ID NO: 21 |
| GAPDH | ACAGCCTCAAGATCATCAGCAAT | SEQ ID NO: 23 |
| | Reverse Primer (5′-3′) | |
| TAGLN | CTGCGCTTTCTTCATAAACC | SEQ ID NO: 10 |
| TAGLN3 | GCTGGGCTTTCCTGTGAAACC | SEQ ID NO: 12 |
| ECAD | AAGAGCACCTTCCATGACAGAC | SEQ ID NO: 14 |
| OCCL | AGGAGAGGTCCATTTGTAGAAGTGA | SEQ ID NO: 16 |
| CTTNB1 | CACTGCCATTTTAGCTCCTTCTTG | SEQ ID NO: 18 |
| VIM | GGATTTCCTCTTCGTGGAGTTTC | SEQ ID NO: 20 |
| FN1 | GATGCAGGTACAGTCCCAGATC | SEQ ID NO: 22 |
| GAPDH | ATGGACTGTGGTCATGAGTCCTT | SEQ ID NO: 24 |

h. Transwell Invasion Assay

Cell invasion assay was performed as described [Shen Y, et al. 2008] using the Transwell filter (pore size, 8.0 μm; 24-well plate; Corning, Inc, Life Sciences). Filters were coated with 1.54 mg/ml Matrigel (BD Biosciences) according to the manufacturer's protocol. Cells were harvested and resuspended in serum-free medium, and 5×10⁵ cells were applied onto the upper chamber of the Transwell filter. The bottom chamber contained 0.6 ml of medium supplemented with 10% fetal bovine serum. Cells were incubated for 40 hours. Cells that did not migrate were removed by cotton swabbing. Cells that invaded to the lower surface of the filter were fixed and stained with 0.25% crystal violet, 3.7% formaldehyde in 80% methanol for 30 minutes at room temperature. The stained cells were extracted with 10% acetic acid, and the absorbance at 595 nm was measured.

i. Clonogenic Survival Assay

Cells were plated at 3×10² per T-25 flask and incubated with complete growth medium for 10 days for HCT116 cells and 14 days for SW480 cells. Colonies were fixed and stained with staining buffer (0.25% crystal violet, 3.7% formaldehyde in 80% methanol) for 30 minutes at room temperature.

j. Anoikis Assay

Anoikis was induced by plating the cells on poly-2-hydroxyethyl methacrylate (polyHEMA; Sigma-Aldrich)-coated culture dishes for 72 hours. Cells were collected by gentle pipetting and either subjected to flow cytometry analysis or replated in regular culture dishes, with attached cells trypsinized and counted at 24 hours.

k. Flow Cytometry

Cells were harvested and washed once with phosphate-buffered saline and resuspended in Annexin-binding buffer (10 mM HEPES, 140 mM NaCl, and 2.5 mM CaCl2, pH 7.4). For each reaction, 1×10⁵ cells were incubated with 10 μg RNase (Sigma-Aldrich), 1 μl of Annexin V conjugated to Alexa Fluor 594 (Molecular Probes), and 0.8 μg of propidium iodide (Invitrogen) for 25 minutes at room temperature. Flow cytometry analysis was carried out using a FACSCalibur flow cytometer (BD Biosciences) with CellQuest software.

I. Statistical Analysis

Values are presented as mean±SD. Comparisons of the means between indicated groups were carried out using Student's t test. Comparisons with clinical and pathologic variables (sex, ethnicity, histologic grade, tumor subsite, and T stage) were made using exact tests for R×C contingency tables. TMA data were analyzed using nonparametric Wilcoxon rank sums test [Kreisberg J I, et al. 2004]. A level of P<0.05 was considered significant.

### D. REFERENCES

Adam P J, Regan C P, Hautmann M B, and Owens G K (2000). Positive- and negative-acting Kruppel-like transcription factors bind a transforming growth factor β control element required for expression of the smooth muscle cell differentiation marker S M22α in vivo. J Biol Chem 275, 37798-37806.

Arnouk H, Merkley M A, Podolsky R H, Stoppler H, Santos C, Alvarez M, Mariategui J, Ferris D, Lee J R, and Dynan W S (in press). Characterization of molecular markers indicative of cervical cancer progression. Proteomics Clin Appl.

Assinder S J, Stanton J A, and Prasad P D (2009). Transgelin: an actin-binding protein and tumour suppressor. Int J Biochem Cell Biol 41, 482-486.

Bhowmick N A, Ghiassi M, Bakin A, Aakre M, Lundquist C A, Engel M E, Arteaga C L, and Moses H L (2001). Transforming growth factor-β1 mediates epithelial to mesenchymal transdifferentiation through a RhoA-dependent mechanism. Mol Biol Cell 12, 27-36.

Birchmeier C, Birchmeier W, Gherardi E, and Vande Woude G F (2003). Met, metastasis, motility and more. Nat Rev Mol Cell Biol 4, 915-925.

Camoretti-Mercado B, Forsythe S M, LeBeau M M, Espinosa R III, Vieira J E, Halayko A J, Willadsen S, Kurtz B, Ober C, Evans G A, et al. (1998). Expression and cytogenetic localization of the human SM22 gene (TAGLN). Genomics 49, 452-457.

Caprioli R M (2008). Perspectives on imaging mass spectrometry in biology and medicine. Proteomics 8, 3679-3680.

Cardoso J, Boer J, Morreau H, and Fodde R (2007). Expression and genomic profiling of colorectal cancer. Biochim Biophys Acta 1775, 103-137.

Chen S, Kulik M, and Lechleider R J (2003). Smad proteins regulate transcriptional induction of the SM22α gene by TGF-β. Nucleic Acids Res 31, 1302-1310.

Chiang, A. C. and Massague, J. (2008) Molecular basis of metastasis. N Engl J Med, 359, 2814-2823.

Christofori, G. (2006) New signals from the invasive front. Nature, 441, 444-450.

Compton, C. C. (2007) Optimal pathologic staging: defining stage II disease. Clin Cancer Res, 13, 6862s-6870s.

Craig D H, Downey C, and Basson M D (2008). SiRNA-mediated reduction of α-actinin-1 inhibits pressure-induced murine tumor cell wound implantation and enhances tumor-free survival. Neoplasia 10, 217-222.

Di Renzo M F, Olivero M, Giacomini A, Porte H, Chastre E, Mirossay L, Nordlinger B, Bretti S, Bottardi S, Giordano S, et al. (1995). Overexpression and amplification of the met/HGF receptor gene during the progression of colorectal cancer. Clin Cancer Res 1, 147-154.

Diep, C. B., Kleivi, K., Ribeiro, F. R., Teixeira, M. R., Lindgjaerde, O. C. and Lothe, R. A. (2006) The order of genetic events associated with colorectal cancer progression inferred from meta-analysis of copy number changes. Genes Chromosomes Cancer, 45, 31-41.

Ellenrieder V, Hendler S F, Boeck W, Seufferlein T, Menke A, Ruhland C, Adler G, and Gress T M (2001). Transforming growth factor β1 treatment leads to an epithelial-mesenchymal transdifferentiation of pancreatic cancer cells requiring extracellular signal-regulated kinase 2 activation. Cancer Res 61, 4222-4228.

Engle S J, Hoying J B, Boivin G P, Ormsby I, Gartside P S, and Doetschman T (1999). Transforming growth factor β1 suppresses nonmetastatic colon cancer at an early stage of tumorigenesis. Cancer Res 59, 3379-3386.

Farrell, C., Crimm, H., Meeh, P., Croshaw, R., Barbar, T., Vandersteenhoven, J. J., Butler, W. and Buckhaults, P. (2008) Somatic mutations to CSMD1 in colorectal adenocarcinomas. Cancer Biol Ther, 7, 609-613.

Fazekas K, Csuka O, Koves I, Raso E, and Timar J (2000). Experimental and clinicopathologic studies on the function of the HGF receptor in human colon cancer metastasis. Clin Exp Metastasis 18, 639-649.

Feng, J., Lawson, M. A. and Melamed, P. (2008) A proteomic comparison of immature and mature mouse gonadotrophs reveals novel differentially expressed nuclear proteins that regulate gonadotropin gene transcription and RNA splicing. Biol Reprod, 79, 546-561.

Fomproix, N. and Percipalle, P. (2004) An actin-myosin complex on actively transcribing genes. Exp Cell Res, 294, 140-148.

Fu Y, Liu H W, Forsythe S M, Kogut P, McConville J F, Halayko A J, Camoretti-Mercado B, and Solway J (2000). Mutagenesis analysis of human SM22: characterization of actin binding. J Appl Physiol 89, 1985-1990.

Fujita S and Sugano K (1997). Expression of c-met proto-oncogene in primary colorectal cancer and liver metastases. Jpn J Clin Oncol 27, 378-383.

Gervaz P, Cerottini J P, Bouzourene H, Hahnloser D, Doan C L, Benhattar J, Chaubert P, Secic M, Gillet M, and Carethers J M (2002). Comparison of microsatellite instability and chromosomal instability in predicting survival of patients with T3N0 colorectal cancer. Surgery 131, 190-197.

Gettemans, J., Van Impe, K., Delanote, V., Hubert, T., Vandekerckhove, J. and De Corte, V. (2005) Nuclear actin-binding proteins as modulators of gene transcription. Traffic, 6, 847-857.

Gimona, M., Kaverina, I., Resch, G. P., Vignal, E. and Burgstaller, G. (2003) Calponin repeats regulate actin filament stability and formation of podosomes in smooth muscle cells. Mol Biol Cell, 14, 2482-2491.

Goldstein, N. S. (2002) Lymph node recoveries from 2427 pT3 colorectal resection specimens spanning 45 years: recommendations for a minimum number of recovered lymph nodes based on predictive probabilities. Am J Surg Pathol, 26, 179-189.

Goldstein, N. S., Sanford, W., Coffey, M. and Layfield, L. J. (1996) Lymph node recovery from colorectal resection specimens removed for adenocarcinoma. Trends over time and a recommendation for a minimum number of lymph nodes to be recovered. Am J Clin Pathol, 106, 209-216.

Greengauz-Roberts, O., Stoppler, H., Nomura, S., Yamaguchi, H., Goldenring, J. R., Podolsky, R. H., Lee, J. R. and Dynan, W. S. (2005) Saturation labeling with cysteine-reactive cyanine fluorescent dyes provides increased sensitivity for protein expression profiling of laser-microdissected clinical specimens. Proteomics, 5, 1746-1757.

Grothey, A. and Ellis, L. M. (2008) Targeting angiogenesis driven by vascular endothelial growth factors using antibody-based therapies. Cancer J, 14, 170-177.

Hanahan, D. and Weinberg, R. A. (2000) The hallmarks of cancer. Cell, 100, 57-70.

Haydon A M and Jass J R (2002). Emerging pathways in colorectal-cancer development. Lancet Oncol 3, 83-88.

Ho J, Kong J W, Choong L Y, Loh M C, Toy W, Chong P K, Wong C H, Wong C Y, Shah N, and Lim Y P (2009). Novel breast cancer metastasis-associated proteins. J Proteome Res 8, 583-594.

Hofmann, W. A., Stojiljkovic, L., Fuchsova, B., Vargas, G. M., Mavrommatis, E., Philimonenko, V., Kysela, K., Goodrich, J. A., Lessard, J. L., Hope, T. J., Hozak, P. and de Lanerolle, P. (2004) Actin is part of pre-initiation complexes and is necessary for transcription by RNA polymerase II. Nat Cell Biol, 6, 1094-1101.

Hsu S, Huang F, Hafez M, Winawer S, and Friedman E (1994). Colon carcinoma cells switch their response to transforming growth factor β1 with tumor progression. Cell Growth Differ 5, 267-275.

Huang, Q., Huang, Q., Chen, W., Wang, L., Lin, W., Lin, J. and Lin, X. (2008) Identification of transgelin as a potential novel biomarker for gastric adenocarcinoma based on proteomics technology. J Cancer Res Clin Oncol, 134, 1219-1227.

Hugo H, Ackland M L, Blick T, Lawrence M G, Clements J A, Williams E D, and Thompson E W (2007). Epithelial-mesenchymal and mesenchymal-epithelial transitions in carcinoma progression. J Cell Physiol 213, 374-383.

Jass, J. R. (2007) Classification of colorectal cancer based on correlation of clinical, morphological and molecular features. Histopathology, 50, 113-130.

Je, H. D. and Sohn, U. D. (2007) SM22alpha is required for agonist-induced regulation of contractility: evidence from SM22alpha knockout mice. Mol Cells, 23, 175-181.

Jemal, A., Siegel, R., Ward, E., Hao, Y., Xu, J., Murray, T. and Thun, M. J. (2008) Cancer statistics, 2008. CA Cancer J Clin, 58, 71-96.

Jensen, L. J., Kuhn, M., Stark, M., Chaffron, S., Creevey, C., Muller, J., Doerks, T., Julien, P., Roth, A., Simonovic, M., Bork, P. and von Mering, C. (2009) STRING 8—a global view on proteins and their functional interactions in 630 organisms. Nucleic Acids Res, 37, D412-416.

Jezequel, P., Campone, M., Roche, H., Gouraud, W., Charbonnel, C., Ricolleau, G., Magrangeas, F., Minvielle, S., Geneve, J., Martin, A. L., Bataille, R. and Campion, L. (2008) A 38-gene expression signature to predict metastasis risk in node-positive breast cancer after systemic adjuvant chemotherapy: a genomic substudy of PACS01 clinical trial. Breast Cancer Res Treat.

Kammula U S, Kuntz E J, Francone T D, Zeng Z, Shia J, Landmann R G, Paty P B, and Weiser M R (2007). Molecular co-expression of the c-Met oncogene and hepatocyte growth factor in primary colon cancer predicts tumor stage and clinical outcome. Cancer Lett 248, 219-228.

Kaplan R N, Riba R D, Zacharoulis S, Bramley A H, Vincent L, Costa C, MacDonald D D, Jin D K, Shido K, Kerns S A, et al. (2005). VEGFR1-positive haematopoietic bone marrow progenitors initiate the pre-metastatic niche. Nature 438, 820-827.

Kokkinos M I, Wafai R, Wong M K, Newgreen D F, Thompson E W, and Waltham M (2007). Vimentin and epithelial-mesenchymal transition in human breast cancer—observations in vitro and in vivo. Cells Tissues Organs 185, 191-203.

Kreisberg J I, Malik S N, Prihoda T J, Bedolla R G, Troyer D A, Kreisberg S, and Ghosh P M (2004). Phosphorylation of Akt (Ser473) is an excellent predictor of poor clinical outcome in prostate cancer. Cancer Res 64, 5232-5236.

Kukalev, A., Nord, Y., Palmberg, C., Bergman, T. and Percipalle, P. (2005) Actin and hnRNP U cooperate for productive transcription by RNA polymerase II. Nat Struct Mol Biol, 12, 238-244.

Lawson, D., Harrison, M. and Shapland, C. (1997) Fibroblast transgelin and smooth muscle SM22alpha are the same protein, the expression of which is down-regulated in many cell lines. Cell Motil Cytoskeleton, 38, 250-257.

Lee, S., Kang, J., Cho, M., Seo, E., Choi, H., Kim, E., Kim, J., Kim, H., Kang, G. Y., Kim, K. P. et al. (2009) Profiling of transcripts and proteins modulated by K-ras oncogene in the lung tissues of K-ras transgenic mice by omics approaches. Int J Oncol, 34, 161-172.

Lehmann K, Janda E, Pierreux C E, Rytomaa M, Schulze A, McMahon M, Hill C S, Beug H, and Downward J (2000). Raf induces TGFβ production while blocking its apoptotic but not invasive responses: a mechanism leading to increased malignancy in epithelial cells. Genes Dev 14, 2610-2622.

Li N, Zhang J, Liang Y, Shao J, Peng F, Sun M, Xu N, Li X, Wang R, Liu S, et al. (2007). A controversial tumor marker: is SM22 a proper biomarker for gastric cancer cells? J Proteome Res 6, 3304-3312.

Li, M., Li, S., Lou, Z., Liao, X., Zhao, X., Meng, Z., Bartlam, M. and Rao, Z. (2008) Crystal structure of human transgelin. J Struct Biol, 162, 229-236.

Lien S C, Usami S, Chien S, and Chiu J J (2006). Phosphatidylinositol 3-kinase/Akt pathway is involved in transforming growth factor-β1-induced phenotypic modulation of 10T1/2 cells to smooth muscle cells. Cell Signal 18, 1270-1278.

Lin, Y., Dynan, W. S., Lee, J. R., Zhu, Z. H. and Schade, R. R. (2009) The current state of proteomics in GI oncology. Dig Dis Sci, 54, 431-457.

Lin, Y., J., B. P., Lee, J. R., Xiong, H., Farrell, C., Podolsky, R. H., Schade, R. R. and Dynan, W. S. (2009) Association of the actin-binding protein transgelin with lymph node metastasis in human colorectal cancer. Neoplasia, In press.

Ma, Y., Qian, Y., Wei, L., Abraham, J., Shi, X., Castranova, V., Harner, E. J., Flynn, D. C. and Guo, L. (2007) Population-based molecular prognosis of breast cancer by transcriptional profiling. Clin Cancer Res, 13, 2014-2022.

Madoz-Gurpide, J., Lopez-Serra, P., Martinez-Torrecuadrada, J. L., Sanchez, L., Lombardia, L. and Casal, J. I. (2006) Proteomics-based validation of genomic data: applications in colorectal cancer diagnosis. Mol Cell Proteomics, 5, 1471-1483.

Medugno L, Florio F, De Cegli R, Grosso M, Lupo A, Costanzo P, and Izzo P (2005). The Kruppel-like zinc-finger protein ZNF224 represses aldolase A gene transcription by interacting with the KAP-1 co-repressor protein. Gene 359, 35-43.

Mikuriya, K., Kuramitsu, Y., Ryozawa, S., Fujimoto, M., Mori, S., Oka, M., Hamano, K., Okita, K., Sakaida, I. and Nakamura, K. (2007) Expression of glycolytic enzymes is increased in pancreatic cancerous tissues as evidenced by proteomic profiling by two-dimensional electrophoresis and liquid chromatography-mass spectrometry/mass spectrometry. Int J Oncol, 30, 849-855.

Molloy, M. P., Brzezinski, E. E., Hang, J., McDowell, M. T. and VanBogelen, R. A. (2003) Overcoming technical variation and biological variation in quantitative proteomics. Proteomics, 3, 1912-1919.

Nair R R, Solway J, and Boyd D D (2006). Expression cloning identifies transgelin (SM22) as a novel repressor of 92-kDa type IV collagenase (MMP-9) expression. J Biol Chem 281, 26424-26436.

Natalwala, A., Spychal, R. and Tselepis, C. (2008) Epithelial-mesenchymal transition mediated tumourigenesis in the gastrointestinal tract. World J Gastroenterol, 14, 3792-3797.

Oba, K., Matsuyama, H., Yoshihiro, S., Kishi, F., Takahashi, M., Tsukamoto, M., Kinjo, M., Sagiyama, K. and Naito, K. (2001) Two putative tumor suppressor genes on chromosome arm 8p may play different roles in prostate cancer. Cancer Genet Cytogenet, 124, 20-26.

Pei, H., Zhu, H., Zeng, S., Li, Y., Yang, H., Shen, L., Chen, J., Zeng, L., Fan, J., Li, X., Gong, Y. and Shen, H. (2007) Proteome analysis and tissue microarray for profiling protein markers associated with lymph node metastasis in colorectal cancer. J Proteome Res, 6, 2495-2501.

Percipalle, P., Fomproix, N., Kylberg, K., Miralles, F., Bjorkroth, B., Daneholt, B. and Visa, N. (2003) An actin-ribonucleoprotein interaction is involved in transcription by RNA polymerase II. Proc Natl Acad Sci USA, 100, 6475-6480.

Portella G, Cumming S A, Liddell J, Cui W, Ireland H, Akhurst R J, and Balmain A (1998). Transforming growth factor β is essential for spindle cell conversion of mouse skin carcinoma in vivo: implications for tumor invasion. Cell Growth Differ 9, 393-404.

Qi, Y., Chiu, J. F., Wang, L., Kwong, D. L. and He, Q. Y. (2005) Comparative proteomic analysis of esophageal squamous cell carcinoma. Proteomics, 5, 2960-2971.

Qiu P, Feng X H, and Li L (2003). Interaction of Smad3 and SRF-associated complex mediates TGF-β1 signals to regulate SM22 transcription during myofibroblast differentiation. J Mol Cell Cardiol 35, 1407-1420.

Qiu P, Ritchie R P, Fu Z, Cao D, Cumming J, Miano J M, Wang D Z, Li H J, and Li L (2005). Myocardin enhances Smad3-mediated transforming growth factor-β3 signaling in a CArG box—independent manner: Smad-binding element is an important cis element for SM22α transcription in vivo. Circ Res 97, 983-991.

Qiu P, Ritchie R P, Gong X Q, Hamamori Y, and Li L (2006). Dynamic changes in chromatin acetylation and the expression of histone acetyltransferases and histone deacetylases regulate the SM22α transcription in response to Smad3-mediated TGFβ1 signaling. Biochem Biophys Res Commun 348, 351-358.

Ryu J W, Kim H J, Lee Y S, Myong N H, Hwang C H, Lee G S, and Yom H C (2003). The proteomics approach to find biomarkers in gastric cancer. J Korean Med Sci 18, 505-509.

Ryu, J. W., Kim, H. J., Lee, Y. S., Myong, N. H., Hwang, C. H., Lee, G. S. and Yom, H. C. (2003) The proteomics approach to find biomarkers in gastric cancer. J Korean Med Sci, 18, 505-509.

Saletti, P. and Cavalli, F. (2006) Metastatic colorectal cancer. Cancer Treat Rev, 32, 557-571.

Seeley E H and Caprioli R M (2008). Molecular imaging of proteins in tissues by mass spectrometry. Proc Natl Acad Sci USA 105, 18126-18131.

Shafer, S. L. and Towler, D. A. (2009) Transcriptional regulation of SM22alpha by Wnt3a: convergence with TGFbeta(1)/Smad signaling at a novel regulatory element. J Mol Cell Cardiol, 46, 621-635.

Shapland, C., Hsuan, J. J., Totty, N. F. and Lawson, D. (1993) Purification and properties of transgelin: a transformation and shape change sensitive actin-gelling protein. J Cell Biol, 121, 1065-1073.

Shen Y, Zhang Y W, Zhang Z X, Miao Z H, and Ding J (2008). hTERT-targeted RNA interference inhibits tumorigenicity and motility of HCT116 cells. Cancer Biol Ther 7, 228-236.

Shields, J. M., Rogers-Graham, K. and Der, C. J. (2002) Loss of transgelin in breast and colon tumors and in RIE-1 cells by Ras deregulation of gene expression through Raf-independent pathways. J Biol Chem, 277, 9790-9799.

Sitek, B., Luttges, J., Marcus, K., Kloppel, G., Schmiegel, W., Meyer, H. E., Hahn, S. A. and Stuhler, K. (2005) Application of fluorescence difference gel electrophoresis saturation labelling for the analysis of microdissected precursor lesions of pancreatic ductal adenocarcinoma. Proteomics, 5, 2665-2679.

Smith, F. M., Gallagher, W. M., Fox, E., Stephens, R. B., Rexhepaj, E., Petricoin, E. F., 3rd, Liotta, L., Kennedy, M. J. and Reynolds, J. V. (2007) Combination of SELDI-TOF-MS and data mining provides early-stage response prediction for rectal tumors undergoing multimodal neoadjuvant therapy. Ann Surg, 245, 259-266.

Stein U, Walther W, Arlt F, Schwabe H, Smith J, Fichtner I, Birchmeier W, and Schlag P M (2009). MACC1, a newly identified key regulator of HGF-MET signaling, predicts colon cancer metastasis. Nat Med 15, 59-67.

Tabor, M. P., van Houten, V. M., Kummer, J. A., Vosjan, M. J., Vlasblom, R., Snow, G. B., Leemans, C. R., Braakhuis, B. J. and Brakenhoff, R. H. (2002) Discordance of genetic alterations between primary head and neck tumors and corresponding metastases associated with mutational status of the TP53 gene. Genes Chromosomes Cancer, 33, 168-177.

Takayama T, Miyanishi K, Hayashi T, Sato Y, and Niitsu Y (2006). Colorectal cancer: genetics of development and metastasis. J Gastroenterol 41, 185-192.

Takeuchi H, Bilchik A, Saha S, Turner R, Wiese D, Tanaka M, Kuo C, Wang H J, and Hoon D S (2003). c-METexpression level in primary colon cancer: a predictor of tumor invasion and lymph node metastases. Clin Cancer Res 9, 1480-1488.

Tse J C and Kalluri R (2007). Mechanisms of metastasis: epithelial-to-mesenchymal transition and contribution of tumor microenvironment. J Cell Biochem 101, 816-829.

Tusher, V. G., Tibshirani, R. and Chu, G. (2001) Significance analysis of microarrays applied to the ionizing radiation response. Proc Natl Acad Sci USA, 98, 5116-5121.

Vartiainen, M. K. (2008) Nuclear actin dynamics—from form to function. FEBS Lett, 582, 2033-2040.

Vartiainen M K, Guettler S, Larijani B, and Treisman R (2007). Nuclear actin regulates dynamic subcellular localization and activity of the SRF cofactorMAL. Science 316, 1749-1752.

Vogelstein, B. and Kinzler, K. W. (2004) Cancer genes and the pathways they control. Nat Med, 10, 789-799.

Vogelstein, B., Fearon, E. R., Hamilton, S. R., Kern, S. E., Preisinger, A. C., Leppert, M., Nakamura, Y., White, R., Smits, A. M. and Bos, J. L. (1988) Genetic alterations during colorectal-tumor development. N Engl J Med, 319, 525-532.

Wang, Z., Jiang, L., Huang, C., Li, Z., Chen, L., Gou, L., Chen, P., Tong, A., Tang, M., Gao, F. et al. (2008) Comparative proteomics approach to screening of potential diagnostic and therapeutic targets for oral squamous cell carcinoma. Mol Cell Proteomics, 7, 1639-1650.

Wei, D., Kanai, M., Huang, S. and Xie, K. (2006) Emerging role of KLF4 in human gastrointestinal cancer. Carcinogenesis, 27, 23-31.

Weinberger P M, Merkley M A, Lee J R, Adam B L, Gourin C G, Podolsky R H, Haffty B G, Papadavid E, Sasaki C, Psyrri A, et al. (in press). Combination proteomic analysis demonstrates molecular similarity of head and neck squamous cell carcinoma arising from different subsites. Arch Otolaryngol.

Yang, Z., Chang, Y. J., Miyamoto, H., Ni, J., Niu, Y., Chen, Z., Chen, Y. L., Yao, J. L., di Sant'Agnese, P. A. and Chang, C. (2007) Transgelin functions as a suppressor via inhibition of ARA54-enhanced androgen receptor transactivation and prostate cancer cell growth. Mol Endocrinol, 21, 343-358.

Yeo, M., Kim, D. K., Park, H. J., Oh, T. Y., Kim, J. H., Cho, S. W., Paik, Y. K. and Hahm, K. B. (2006) Loss of transgelin in repeated bouts of ulcerative colitis-induced colon carcinogenesis. Proteomics, 6, 1158-1165.

Young, K. G. and Kothary, R. (2005) Spectrin repeat proteins in the nucleus. Bioessays, 27, 144-152.

Yu, H., Konigshoff, M., Jayachandran, A., Handley, D., Seeger, W., Kaminski, N. and Eickelberg, O. (2008) Transgelin is a direct target of TGF-beta/Smad3-dependent epithelial cell migration in lung fibrosis. Faseb J, 22, 1778-1789.

Zhou, W., Goodman, S. N., Galizia, G., Lieto, E., Ferraraccio, F., Pignatelli, C., Purdie, C. A., Piris, J., Morris, R., Harrison, D. J. et al. (2002) Counting alleles to predict recurrence of early-stage colorectal cancers. Lancet, 359, 219-225.

### E. SEQUENCES

What is claimed is:

1. A method of determining the metastatic potential of a colorectal cancer in a subject, the method comprising assaying for the levels of transgelin in a sample from the subject, wherein an increase in transgelin levels in the sample compared to a control level is an indication that the colorectal cancer in the subject is metastatic.

2. The method of claim 1, wherein cancer cells were not previously detected in the lymph nodes of the subject.

3. The method of claim 1, wherein the sample is a tumor biopsy.

4. The method of claim 1, wherein the method comprises detecting transgelin using an antibody that specifically binds transgelin.

5. The method of claim 1, wherein the method comprises detecting transgelin using a primer or probe that selectively binds TAGLN mRNA.

6. A method of selecting a therapy for a subject diagnosed with a colorectal cancer, the method comprising assaying for the levels of transgelin in a sample from the subject, wherein an increase in transgelin levels in the sample compared to a control level is an indication that the therapy selected comprises surgical resection and adjuvant treatment.

7. A method of treating metastatic colorectal cancer in a subject, comprising administering to the subject an inhibitor of transgelin activity.

8. The method of claim 7, wherein the transgelin inhibitor is a functional nucleic acid.

9. A method of identifying an agent for use in treating colorectal cancer, comprising contacting a sample comprising transgelin with a candidate agent and assaying for transgelin activity in the sample, wherein a decrease in transgelin activity in the sample is an indication that the candidate agent is an effective agent for use in treating colorectal cancer.

10. The method of claim 9, wherein method comprises cell-based assay.

11. The method of claim 10, wherein the cell is a colorectal cancer cell line.
